SEQUENCE LISTING (63 sequences)

SEQ ID NO: 1 - DNA, 1326 bp, Artificial
  HC of humanized antiAbeta_13C13_IgG4 mAb
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 2 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - DNA, 660 bp, Artificial
  LC of humanized antiAbeta_13C13_D260X mAb
gagatcgtgatgacccaaactccactctccctgcctgtcagtcttggagatagagcctccatctcttgcagatctggtcagagccttgtgcacagtaatggaaacacctatctgcattggtacctgcagaagccaggccagtctccaaagctcctgatctatacagtttccaaccgattttctggggtcccggacaggttcagtggcagtggatcagggtcagatttcacactcaccatcagcagagtggaggctgaggatctgggagtttatttctgctctcaaaatacatttgttccttggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 4 - protein, 219 aa, Artificial
  Synthetic Construct
EIVMTQTPLSLPVSLGDRASISCRSGQSLVHSNGNTYLHWYLQKPGQSPKLLIYTVSNRFSGVPDRFSGSGSGSDFTLTISRVEAEDLGVYFCSQNTFVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 345 bp, Artificial
  Humanised sequence
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagc

SEQ ID NO: 6 - protein, 115 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSS

SEQ ID NO: 7 - DNA, 339 bp, Artificial
  Humanised sequence
gagatcgtgatgacccaaactccactctccctgcctgtcagtcttggagatagagcctccatctcttgcagatctggtcagagccttgtgcacagtaatggaaacacctatctgcattggtacctgcagaagccaggccagtctccaaagctcctgatctatacagtttccaaccgattttctggggtcccggacaggttcagtggcagtggatcagggtcagatttcacactcaccatcagcagagtggaggctgaggatctgggagtttatttctgctctcaaaatacatttgttccttggacgttcggtggaggcaccaagctggaaatcaaacgt

SEQ ID NO: 8 - protein, 113 aa, Artificial
  Synthetic Construct
EIVMTQTPLSLPVSLGDRASISCRSGQSLVHSNGNTYLHWYLQKPGQSPKLLIYTVSNRFSGVPDRFSGSGSGSDFTLTISRVEAEDLGVYFCSQNTFVPWTFGGGTKLEIKR

SEQ ID NO: 9 - DNA, 33 bp, Mus sp.
  CDS (1)..(33)
tccggctacacattcactgattatgctatgcac

SEQ ID NO: 10 - protein, 11 aa, Mus sp.
SGYTFTDYAMH

SEQ ID NO: 11 - DNA, 30 bp, Mus sp.
  CDS (1)..(30)
gttattagtactaagtatggtaagacaaac

SEQ ID NO: 12 - protein, 10 aa, Mus sp.
VISTKYGKTN

SEQ ID NO: 13 - DNA, 18 bp, Mus sp.
  CDS (1)..(18)
ggggacgatggttattcc

SEQ ID NO: 14 - protein, 6 aa, Mus sp.
GDDGYS

SEQ ID NO: 15 - DNA, 48 bp, Mus sp.
  CDS (1)..(48)
agatctggtcagagccttgtgcacagtaatggaaacacctatctgcat

SEQ ID NO: 16 - protein, 16 aa, Mus sp.
RSGQSLVHSNGNTYLH

SEQ ID NO: 17 - DNA, 24 bp, Mus sp.
  CDS (1)..(24)
acagtttccaaccgattttctggg

SEQ ID NO: 18 - protein, 8 aa, Mus sp.
TVSNRFSG

SEQ ID NO: 19 - DNA, 27 bp, Mus sp.
  CDS (1)..(27)
tctcaaaatacatttgttccttggacg

SEQ ID NO: 20 - protein, 9 aa, Mus sp.
SQNTFVPWT

SEQ ID NO: 21 - DNA, 660 bp, Artificial
  Humanised sequence
gagatcgtgatgacccaaactccactctccctgcctgtcagtcttggagatagagcctccatctcttgcagatctggtcagagccttgtgcacagtaataccaacacctatctgcattggtacctgcagaagccaggccagtctccaaagctcctgatctatacagtttccaaccgattttctggggtcccggacaggttcagtggcagtggatcagggtcagatttcacactcaccatcagcagagtggaggctgaggatctgggagtttatttctgctctcaaaatacatttgttccttggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 22 - protein, 219 aa, Artificial
  Synthetic Construct
EIVMTQTPLSLPVSLGDRASISCRSGQSLVHSNTNTYLHWYLQKPGQSPKLLIYTVSNRFSGVPDRFSGSGSGSDFTLTISRVEAEDLGVYFCSQNTFVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 339 bp, Artificial
  Humanised sequence
gagatcgtgatgacccaaactccactctccctgcctgtcagtcttggagatagagcctccatctcttgcagatctggtcagagccttgtgcacagtaataccaacacctatctgcattggtacctgcagaagccaggccagtctccaaagctcctgatctatacagtttccaaccgattttctggggtcccggacaggttcagtggcagtggatcagggtcagatttcacactcaccatcagcagagtggaggctgaggatctgggagtttatttctgctctcaaaatacatttgttccttggacgttcggtggaggcaccaagctggaaatcaaacgt

SEQ ID NO: 24 - protein, 113 aa, Artificial
  Synthetic Construct
EIVMTQTPLSLPVSLGDRASISCRSGQSLVHSNTNTYLHWYLQKPGQSPKLLIYTVSNRFSGVPDRFSGSGSGSDFTLTISRVEAEDLGVYFCSQNTFVPWTFGGGTKLEIKR

SEQ ID NO: 25 - DNA, 1326 bp, Artificial
  Humanised sequence
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgagggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtggacgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 26 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDEGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - DNA, 345 bp, Artificial
  Humanised sequence
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgagggttattcctggggtcaaggaacctcagtcaccgtctccagc

SEQ ID NO: 28 - protein, 115 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDEGYSWGQGTSVTVSS

SEQ ID NO: 29 - DNA, 18 bp, Mus sp.
  CDS (1)..(18)
ggggacgagggttattcc

SEQ ID NO: 30 - protein, 6 aa, Mus sp.
GDEGYS

SEQ ID NO: 31 - DNA, 48 bp, Mus sp.
  CDS (1)..(48)
agatctggtcagagccttgtgcacagtaataccaacacctatctgcat

SEQ ID NO: 32 - protein, 16 aa, Mus sp.
RSGQSLVHSNTNTYLH

SEQ ID NO: 33 - DNA, 1221 bp, homo sapiens
atgattcacaccaacctgaagaaaaagttcagctgctgcgtcctggtctttcttctgtttgcagtcatctgtgtgtggaaggaaaagaagaaagggagttactatgattcctttaaattgcaaaccaaggaattccaggtgttaaagagtctggggaaattggccatggggtctgattcccagtctgtatcctcaagcagcacccaggacccccacaggggccgccagaccctcggcagtctcagaggcctagccaaggccaaaccagaggcctccttccaggtgtggaacaaggacagctcttccaaaaaccttatccctaggctgcaaaagatctggaagaattacctaagcatgaacaagtacaaagtgtcctacaaggggccaggaccaggcatcaagttcagtgcagaggccctgcgctgccacctccgggaccatgtgaatgtatccatggtagaggtcacagattttcccttcaatacctctgaatgggagggttatctgcccaaggagagcattaggaccaaggctgggccttggggcaggtgtgctgttgtgtcgtcagcgggatctctgaagtcctcccaactaggcagagaaatcgatgatcatgacgcagtcctgaggtttaatggggcacccacagccaacttccaacaagatgtgggcacaaaaactaccattcgcctgatgaactctcagttggttaccacagagaagcgcttcctcaaagacagtttgtacaatgaaggaatcctaattgtatgggacccatctgtataccactcagatatcccaaagtggtaccagaatccggattataatttctttaataactacaagacttatcgtaagctgcaccccaatcagcccttttacatcctcaagccccagatgccttgggagctatgggacattcttcaagaaatctccccagaagagattcagccaaaccccccatcctctgggatgcttggtatcatcatcatgatgacgctgtgtgaccaggtggatatttatgagttcctcccatccaagcgcaagactgacgtgtgctactactaccagaagttcttcgatagtgcctgcacgatgggtgcctaccacccgctgctctatgagaagaatttggtgaagcatctcaaccagggcacagatgaggacatctacctgcttggaaaagccacactgcctggcttccggaccattcactgctaa

SEQ ID NO: 34 - protein, 406 aa, homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 35 - DNA, 1197 bp, homo sapiens
atgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcatccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctag

SEQ ID NO: 36 - protein, 398 aa, homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 37 - DNA, 1374 bp, mus musculus
atggaatgcagctgggtctttctctttctggtagcaacagctacaggtgtgcactcccaggtccagctgcagcagtctgggcctgagctggtgaggcctggggtctcagtgaagatttcctgcaagggttccggctacacattcactgattatgctatgcactgggtgaagcagagtcatgcaaagagtctagagtggattggagttattagtactaagtatggtaagacaaactacaaccagaagtttaagggcaaggccacaatgactgttgacaaatcctccagcacagcctatatggagcttgccagattgacatctgaggattctgccatctattactgtgcaagaggggacgatggttattcctggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 38 - protein, 438 aa, mus musculus
QVQLQQSGPELVRPGVSVKISCKGSGYTFTDYAMHWVKQSHAKSLEWIGVISTKYGKTNYNQKFKGKATMTVDKSSSTAYMELARLTSEDSAIYYCARGDDGYSWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 39 - DNA, 717 bp, mus musculus
atgaagttgcctgttaggctgttggtgctgatgttctggattcctgcttccagcagtgatgttgtgatgacccaaactccactctccctgcctgtcagtcttggagatcaagcctccatctcttgcagatctggtcagagccttgtacacagtaatggaaacacctatttacattggtacctgcagaagccaggccagtctccaaagctcctgatctatacagtttccaaccgattttctggggtcccggacaggttcagtggcagtggatcagggtcagatttcacactcaagatcagcagagtggaggctgaggatctgggagtttatttctgctctcaaaatacatttgttccttggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 40 - protein, 219 aa, mus musculus
DVVMTQTPLSLPVSLGDQASISCRSGQSLVHSNGNTYLHWYLQKPGQSPKLLIYTVSNRFSGVPDRFSGSGSGSDFTLKISRVEAEDLGVYFCSQNTFVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - DNA, 1374 bp, mus musculus
atggaatgcagctgggtctttctctttctggtagcaacagctacaggtgtgcactcccaggtccagctgcagcagtctgggcctgagctggtgaggcctggggtctcagtgaagatttcctgcaagggttccggctacacattcactgattatgctatgcactgggtgaagcagagtcatgcaaagagtctagagtggattggagttattagtactaagtatggtaagacaaactacaaccagaagtttaagggcaaggccacaatgactgttgacaaatcctccagcacagcctatatggagcttgccagattgacatctgaggattctgccatctattactgtgcaagaggggacgatggttattcctggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcgcccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 42 - protein, 438 aa, mus musculus
QVQLQQSGPELVRPGVSVKISCKGSGYTFTDYAMHWVKQSHAKSLEWIGVISTKYGKTNYNQKFKGKATMTVDKSSSTAYMELARLTSEDSAIYYCARGDDGYSWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIAPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 43 - DNA, 1374 bp, mus musculus
atggaatgcagctgggtctttctctttctggtagcaacagctacaggtgtgcactcccaggtccagctgcagcagtctgggcctgagctggtgaggcctggggtctcagtgaagatttcctgcaagggttccggctacacattcactgattatgctatgcactgggtgaagcagagtcatgcaaagagtctagagtggattggagttattagtactaagtatggtaagacaaactacaaccagaagtttaagggcaaggccacaatgactgttgacaaatcctccagcacagcctatatggagcttgccagattgacatctgaggattctgccatctattactgtgcaagaggggacgatggttattcctggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggcagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 44 - protein, 438 aa, mus musculus
QVQLQQSGPELVRPGVSVKISCKGSGYTFTDYAMHWVKQSHAKSLEWIGVISTKYGKTNYNQKFKGKATMTVDKSSSTAYMELARLTSEDSAIYYCARGDDGYSWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVADISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 45 - DNA, 1374 bp, mus musculus
atggaatgcagctgggtctttctctttctggtagcaacagctacaggtgtgcactcccaggtccagctgcagcagtctgggcctgagctggtgaggcctggggtctcagtgaagatttcctgcaagggttccggctacacattcactgattatgctatgcactgggtgaagcagagtcatgcaaagagtctagagtggattggagttattagtactaagtatggtaagacaaactacaaccagaagtttaagggcaaggccacaatgactgttgacaaatcctccagcacagcctatatggagcttgccagattgacatctgaggattctgccatctattactgtgcaagaggggacgatggttattcctggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagccatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggttga

SEQ ID NO: 46 - protein, 438 aa, mus musculus
QVQLQQSGPELVRPGVSVKISCKGSGYTFTDYAMHWVKQSHAKSLEWIGVISTKYGKTNYNQKFKGKATMTVDKSSSTAYMELARLTSEDSAIYYCARGDDGYSWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 47 - DNA, 1326 bp, Artificial
  HC of humanized antiAbeta_13C13_ IgG4_D260A mAb
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtggccgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 48 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - DNA, 1326 bp, Artificial
  HC of humanized antiAbeta_13C13_ IgG4_D260G mAb
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtgggcgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 50 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVGVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - DNA, 1326 bp, Artificial
  HC of humanized antiAbeta_13C13_IgG4_D260L mAb
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtgctggtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 52 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVLVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 53 - DNA, 1326 bp, Artificial
  HC of humanized antiAbeta_13C13_IgG4_D260K mAb
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtgaaggtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 54 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVKVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - DNA, 1326 bp, Artificial
  HC of humanized antiAbeta_13C13_IgG4_D260S mAb for expression
gaggtccagctgcagcagtctgggcctgaggtggtgaagcctggggtctcagtgaagatttcctgcaagggttccggctacacattcactgattatgctatgcactgggtgaagcagagtcctggcaagagtctggagtggattggagttattagtactaagtatggtaagacaaactacaaccccagctttcagggccaggccacaatgactgttgacaaatcctccagcacagcctatatggagcttgccagcttgaaggcctccgattctgccatctattactgtgcaagaggggacgatggttattcctggggtcaaggaacctcagtcaccgtctccagcgcttctaccaagggcccttccgtgttccctctggccccttgctcccggtccacctccgagtccaccgccgctctgggctgcctggtgaaggactacttccctgagcctgtgaccgtgtcctggaactctggcgccctgacctccggcgtgcacaccttccctgccgtgctgcagtcctccggcctgtactccctgtcctccgtggtgaccgtgccttcctcctccctgggcaccaagacctacacctgtaacgtggaccacaagccttccaacaccaaggtggacaagcgggtggagtccaagtacggccctccttgccctccctgccctgcccctgagttcgagggcggacctagcgtgttcctgttccctcctaagcctaaggacaccctgatgatctcccggacccctgaggtgacctgtgtggtggtgagcgtgtcccaggaggaccctgaggtccagttcaactggtacgtggacggcgtggaggtgcacaacgccaagaccaagcctcgggaggagcagttcaattccacctaccgggtggtgtctgtgctgaccgtgctgcaccaggactggctgaacggcaaagaatacaagtgtaaggtctccaacaagggcctgccctcctccatcgagaaaaccatctccaaggccaagggccagcctagggagcctcaggtgtacaccctgcctcctagccaggaagagatgaccaagaaccaggtgtccctgacctgtctggtgaagggcttctacccttccgacatcgccgtggagtgggagtccaacggccagcctgagaacaactacaagaccacccctcctgtgctggactccgacggctccttcttcctgtactccaggctgaccgtggacaagtcccggtggcaggagggcaacgtcttttcctgctccgtgatgcacgaggccctgcacaaccactacacccagaagtccctgtccctgtctctgggctga

SEQ ID NO: 56 - protein, 441 aa, Artificial
  Synthetic Construct
EVQLQQSGPEVVKPGVSVKISCKGSGYTFTDYAMHWVKQSPGKSLEWIGVISTKYGKTNYNPSFQGQATMTVDKSSSTAYMELASLKASDSAIYYCARGDDGYSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVSVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTWVVVDVSHEDPEVQFNWYVDGVEVHNAKTKPREEQFNSTFCVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 327 aa, Homo sapiens; identical to SEQ ID NO: 57

SEQ ID NO: 61 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 62 - protein, 330 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 63 - protein, 330 aa, Mus musculus
ATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

(19) United States
(12) Patent Application Publication
(10) Pub. No.: US 2014/0046032 A1
(43) Pub. Date: Feb. 13, 2014

(54) METHOD OF PRODUCTION OF SIALYLATED ANTIBODIES

(71) Applicants: Francis Blanche, Paris (FR); Beatrice Cameron, Paris (FR); Bruno Genet, Paris (FR); Fabienne Soubrier, Paris (FR)

(72) Inventors: Francis Blanche, Paris (FR); Beatrice Cameron, Paris (FR); Bruno Genet, Paris (FR); Fabienne Soubrier, Paris (FR)

(73) Assignee: SANOFI, Paris (FR)

(21) Appl. No.: 14/000,035

(22) PCT Filed: Feb. 23, 2012
(86) PCT No.: PCT/EP12/53065

§ 371 Date: Oct. 29, 2013

(30) Foreign Application Priority Data
Feb. 24, 2011 (EP) 11305200.5
Sep. 1, 2011 (EP) 11306090.9

(51) Int. Cl. C07K 16/18

(52) U.S. Cl. CPC C07K 16/18
USPC 530/387.3; 435/69.6

(57) ABSTRACT

The present invention relates to a method for producing an IgG antibody, wherein at least 80% of the said antibody comprises a complex, bi-antennary oligosaccharide, which contains two sialic acid residues, attached to the Fc domain of the antibody. The said method comprises the steps of introducing a mutation in the Fc domain of the antibody, and expressing the mutant antibody in a cell which expresses a galactosyltransferase and a sialyltransferase activity.

### INTRODUCTION

Alzheimer disease (AD) is a progressive neurodegenerative disease affecting a large proportion of the aged population. Beta-Amyloid (Aβ) peptides are thought to be a causative agent through the formation of insoluble Aβ peptide fibrils and deposition of these fibrils to form amyloid plaques (Tanzi and Bertram, Cell, 120: 545-555, 2005). The formation of such plaques within the area of the brain critical for memory and other cognitive functions is thought to lead to dementia associated with this disease (see Selkoe, J. Neuropathol. Exp. Neurol. 53: 438-447, 1994). Aβ is a fragment from a larger protein called amyloid precursor protein (APP), a transmembrane protein that penetrates through the neuron's membrane. In the case of AD, the normal soluble Aβ (sAβ) peptide is converted into oligomeric/fibrillar Aβ. Neuronal toxicity may thus reside in the large molecular weight fibrils which are formed via aggregation of sAβ into insoluble fibrils and, subsequently, the fibril incorporation into amyloid plaques.

Various treatments have been forwarded in attempts to prevent formation of Aβ peptide. Currently, the greatest hope for an intervention that will significantly impact disease progression comes from immunotherapy (Brody and Holtzman, Annu Rev Neurosci, 31: 175-193, 2008; Winiewski and Konietzko, Lancet Neurol, 7: 805-811, 2008; Winiewski and Boutajangout, Brain Struct Funct, 214: 201-218, 2010). Immunotherapy treatment encompasses both the administration of antibodies recognizing specific forms of Aβ (see e.g. WO 2007/068412, WO 2009/065054, WO 2009/048538, WO 2009/052125, WO 2009/074583, EP 2 224 000 A1), as well as immunization with Aβ peptide antigens (see e.g. EP 2 226 081 A1). For example, antibodies directed against the N-terminus of Aβ have been described (U.S. Pat. Nos. 6,761,888 and 6,750,324; Brody and Holtzman, Annu Rev Neurosci, 31: 175-193, 2008); these antibodies can prevent or reverse aggregation of Aβ fibrils. U.S. Pat. No. 7,179,463 discloses a method of treating Alzheimer's disease by administering an antibody raised against a protofibril consisting of the Arctic mutation within the Aβ peptide coding region. No exemplification of raised antibodies are presented in the specification and no comparison as to affinity for low molecular weight forms of Aβ peptide are presented. Moreover, adverse events such as microhaemorraghe and vasogenic oedema have been reported following treatment with some of these antibodies, either in preclinical or clinical trials (Winiewski and Konietzko, Lancet Neurol, 7: 805-811, 2008; Weller et al., Alzheimers Res Ther, 1(2): 6).

New humanized antibodies specific for the protofibrillar form of the Aβ peptide have recently been described (WO 2010/130946). These antibodies recognize only senile plaques, but not diffuse deposits of Aβ peptide, as demonstrated by immunochemistry on Alzheimer's patient's brain samples. In addition, the said humanized antibodies are capable of inducing a diminution of the amyloid plaques.

During the past 15 years a variety of inflammatory proteins has been identified in the brains of patients with AD postmortem. There is now considerable evidence that in AD the deposition of amyloid-β (Aβ) protein precedes a cascade of events that ultimately leads to a local “brain inflammatory response.” It is thus particularly important that therapeutic antibodies for treating AD do not trigger an additional inflammatory reaction.

It is well established that high doses of monomeric immunoglobulin G (IgG) purified from pooled human plasma, so called intravenous immunoglobulin or IVIG, confer anti-inflammatory activity through interactions mediated by its Fc fragment (Samuelsson et al., Science, 291: 484-486, 2001; Kaneko et al., J. Exp. Med. 203: 789-797, 2006). Thus, while Fc-FcyR interactions are responsible for the pro-inflammatory properties of immune complexes and cytotoxic antibodies, IVIG and its Fc fragments are anti-inflammatory and are widely used to suppress inflammatory diseases. Glycosylation, and more specifically sialylation (Kanuko et al., Science, 313: 670-673, 2006), of IgG appears to be crucial for regulation of cytotoxicity and inflammatory potential of IgG: a sialylated recombinant human IgG Fc-portion is sufficient for the anti-inflammatory effect of IVIG (Anthony et al., Science, 320: 373-376, 2008; WO 2007/117505). The linkage between the terminal sialic acid and the penultimate galactose appears to be crucial for the said anti-inflammatory activity (Anthony et al., Science, 320: 373-376, 2008; Anthony et al., Proc Natl Acad Sci U.S.A., 105: 19571-19578, 2008; WO 2007/117505).

Optimizing sialylation of therapeutic antibodies is thus an important factor in improving the treatment of AD. Indeed, using homogeneously-, fully-sialylated antibodies in such a treatment would help minimizing the risks of triggering an adverse inflammatory reaction. It would thus be advantageous to have a method for producing recombinant therapeutic antibodies which are homogeneously and fully sialylated. Moreover, a key feature and challenge for the industry in the production of recombinant antibodies is the optimization of productivity, cost, homogeneity, and antibody activity. In particular, it is known that glycosylation is a key issue in the production of high yields of homogeneous and potent recombinant therapeutic antibodies which poses a series of critical problems for the production of recombinant therapeutic antibodies. Each current production cell line offers a series of different challenges and problems which are largely due to the complexity and species, tissue and site specificity of the glycosylation (see e.g., Jefferis, Biotechnol Prog, 21(1): 11-16, 2005). It is thus necessary that the said method ensures the production of recombinant therapeutic antibodies which are homogeneously and fully sialylated with a productivity high enough for ensuring preclinical and clinical trials.

However, the methods of the prior art only yield antibodies which are either heterogeneously or partially sialylated and/or in quantities too low for use in clinical trials. For example, cell lines expressing exogenous galactosyltransferase and/or sialyltransferase activities were used to produce glycoproteins. However, high expression levels of these enzymes are necessary for obtaining suitable levels of sialylation. In that case, though, the productivity of the cell line is dramatically decreased, which means that it is unsuitable for use as a host cell for production of recombinant therapeutic antibodies. Galactosylation and/or sialylation reactions have also been carried out in vitro. The yields were, however, too low to allow preparation of enough fully-sialylated antibody for in vivo testing. This was not improved by selective enrichment of sialylated antibodies on a lectin-affinity column. Alternatively, mutations have been introduced into the Fc domain of the produced antibody. Alanine residues were thus been introduced at various positions in the Fc domain of IgG3 antibodies. The resulting increase in sialylation was only modest, though, with no more than 30% of disialylated N-glycans obtained in the best of cases (Lund et al., J. Immunol., 157: 4963-4969, 1996; Weikert et al., Nature Biotech., 17: 1116-1121, 1999; Shields et al., J. Biol. Chem., 276(1): 6591-6604, 2001; Jassal et al., Biochem Biophys Res Commun., 286(2): 243-249, 2001; Scallon et al., Mol. Immunol., 44: 1524-1534, 2007; Baudino et al., J. Immunol., 181: 6664-6669, 2008; Hossler et al., Glycobiology, 19(9): 936-949, 2009; WO 2007/048122; WO 2008/057634; WO 2008/065543; WO 2009/079382; WO 2010/109010).

Thus there is still a need for a method for high-level production of antibodies displaying fully-sialylated N-glycans.

### SUMMARY OF THE INVENTION

The methods of the prior art do not allow for the production of extensively sialylated antibodies in amounts consistent with the development of a pharmaceutical product. It has been observed by the inventors that expression of an IgG antibody in a cell line overexpressing a β galactosyltransferase and/or a sialyltransferase yields sialylated antibody only in conditions of very low productivity. Likewise, expression in a regular cell line of an antibody mutated in the Fc domain yields an antibody composition with a very heterogeneous sialylation pattern.

The present inventors have now shown that it is possible to obtain high yields of extensively sialylated IgG antibodies by expressing an antibody carrying a mutation in its Fc domain in a host cell which expresses a β galactosyltransferase and a sialyltransferase activity. The antibodies obtained by the method of the invention present homogeneous glycoforms, said glycoforms comprising N-glycans which are essentially of the complex, bi-antennary form, and wherein both branches of the oligosaccharide carry a sialic acid residue.

According to the invention, “extensively sialylated” means that at least 80%, preferably at least 85%, more preferably at least 90%, even more preferably at least 95%, still most preferably at least 97% or most preferably at least 99% of the N-glycans carried by the Fc domain of the antibodies comprise 2 sialic acid residues by oligosaccharide chain.

A first aspect of the invention pertains to a method for producing an IgG antibody, wherein at least 80% of the said antibody comprises a complex, bi-antennary oligosaccharide, which contains two sialic acid residues, attached to each Fc domain of the antibody, said method comprising the steps of:

a) introducing a mutation in the said Fc domain of the said antibody, and
b) expressing the mutant antibody obtained in step a) in a cell line expressing a β-galactosyltransferase and a sialyltransferase activity.

In a specific embodiment, the β-galactosyltransferase is a β-1,4-galactosyltransferase and the sialyltransferase is a α-2,6sialyltransferase. In another specific embodiment, the β-1,4-galactosyltransferase is encoded by the polynucleotide sequence represented by SEQ ID NO: 35 and the α-2,6sialyltransferase is encoded by the polynucleotide sequence represented by SEQ ID NO: 33. In another specific embodiment, the said sialic acid residues are linked to the antibody through an α-2,6-linkage.

In another specific embodiment, the antibody is a monoclonal antibody. In another specific embodiment, the antibody is a humanized antibody.

In another specific embodiment, the said mutation affects an amino acid selected from the group consisting of F243, V264, and D265. In another specific embodiment, the said mutation is selected from the group consisting of F243A, V264A, and D265A. In another specific embodiment, the said mutation is D265A.

In another specific embodiment, the said antibody comprises an IgG4 Fc domain.

In another specific embodiment, the said antibody binds specifically the protofibrillar form of peptide Aβ. In another specific embodiment, the said antibody has at least one CDR coded by a polynucleotide having a sequence identical to a sequence selected from SEQ ID NOs: 9, 11, 13, 15, 17 and 19, or having a sequence differing from one of the said sequences SEQ ID NOs: 9, 11, 13, 15, 17 and 19, by 1, 2, 3, 4, or 5 nucleotides. In another specific embodiment, the said antibody has at least one CDR displaying a sequence identical to one sequence selected from SEQ ID NOs: 10, 12, 14, 16, 18, and 20. In another specific embodiment, the said antibody has at least one CDR differing from the said sequences by 1 or 2 amino acid residues, while retaining its binding specificity. In another specific embodiment, the said antibody comprises the CDRs encoded by the nucleotide sequences SEQ ID NOs: 9, 11, 13, 15, 17, and 19, or by sequences differing only by 1, 2, 3, 4, or 5 nucleotides from the said sequences SEQ ID NOs: 9, 11, 13, 15, 17, and 19. In another specific embodiment, the said antibody comprises 6 CDRs having sequences identical to the sequences represented by SEQ ID NOs: 10, 12, 14, 16, 18, and 20. In another specific embodiment, the said antibody comprises the CDRs encoded by the nucleotide sequences SEQ ID NOs: 9, 11, 13, 31, 17, and 19, or by sequences differing only by 1, 2, 3, 4, or 5 nucleotides from the said sequences SEQ ID NOs: 9, 11, 13, 31, 17, and 19. In another specific embodiment, the said antibody comprises 6 CDRs having sequences identical to the sequences represented by SEQ ID NOs: 10, 12, 14, 32, 18, and 20. In another specific embodiment, the said antibody comprises the CDRs encoded by the nucleotide sequences SEQ ID NOs: 9, 11, 29, 31, 17, and 19, or by sequences differing only by 1, 2, 3, 4, or 5 nucleotides from the said sequences SEQ ID NOs: 9, 11, 29, 31, 17, and 19. In another specific embodiment, the said antibody comprises 6 CDRs having sequences identical to the sequences represented by SEQ ID NOs: 10, 12, 30, 32, 18, and 20. In another specific embodiment, the said antibody comprises a V_{H} encoded by a polynucleotide sequence displaying at least 80% identity with the sequence represented by SEQ ID NO: 5 or the sequence represented by SEQ ID NO: 27. In another specific embodiment, the said antibody comprises a V_{H} having a sequence having at least 80% identity with the sequence represented by SEQ ID NO: 6 or the sequence represented by SEQ ID NO: 28. In another specific embodiment, the said antibody V_{L} encoded by a polynucleotide sequence displaying at least 80% identity with the sequence represented by SEQ ID NO: 7 or the sequence represented by SEQ ID NO: 23. In another specific embodiment, the said antibody comprises a V_{L} having a sequence having at least 80% identity with the sequence represented by SEQ ID NO: 8 or the sequence represented by SEQ ID NO: 24. In another specific embodiment, the said antibody comprises a V_{H} encoded by the polynucleotide sequence represented by SEQ ID NO: 5 or the polynucleotide sequence represented by SEQ ID NO: 27. In another specific embodiment, the said antibody comprises a V_{H} having the sequence represented by SEQ ID NO: 6 or the sequence represented by SEQ ID NO: 28. In another specific embodiment, the said antibody V_{L} encoded by the polynucleotide sequence represented by SEQ ID NO: 7 or the polynucleotide sequence represented by SEQ ID NO: 23. In another specific embodiment, the said antibody comprises a V_{L} having the sequence represented by SEQ ID NO: 8 or by SEQ ID NO: 24. In another specific embodiment, the said antibody comprises the sequences encoded by the polynucleotide sequences SEQ ID NOs: 5 & 7. In another specific embodiment, the said antibody comprises the amino acid sequences represented by SEQ ID NOs: 6 & 8. In another specific embodiment, the said antibody comprises the sequences encoded by the polynucleotide sequences SEQ ID NOs: 5 & 23. In another specific embodiment, the said antibody comprises the amino acid sequences represented by SEQ ID NOs: 6 & 24. In another specific embodiment, the said antibody comprises the sequences encoded by the polynucleotide sequences SEQ ID NOs: 27 & 23. In another specific embodiment, the said antibody comprises the amino acid sequences represented by SEQ ID NOs: 28 & 24. In another specific embodiment, the said antibody comprises a heavy chain encoded by a polynucleotide sequence having at least 80% identity with a sequence represented by SEQ ID NO: 1 or SEQ ID NO: 25. In another specific embodiment, the said antibody comprises a heavy chain having an amino acid sequence with at least 80% identity with a sequence represented by SEQ ID NO: 2 or SEQ ID NO: 26. In another specific embodiment, the said antibody comprises a light chain encoded by a polynucleotide sequence having at least 80% identity with a sequence represented by SEQ ID NO: 3 or SEQ ID NO: 21. In another specific embodiment, the said antibody comprises a light chain having an amino acid sequence with at least 80% identity with a sequence represented by SEQ ID NO: 4 or SEQ ID NO: 22. In another specific embodiment, the said antibody comprises the sequences encoded by the polynucleotide sequences represented by SEQ ID NOs: 1 & 3. In another specific embodiment, the said antibody has the amino acid sequences represented by SEQ ID NOs: 2 & 4. In another specific embodiment, the said antibody comprises the sequences encoded by the polynucleotide sequences represented by SEQ ID NOs: 1 & 21. In another specific embodiment, the said antibody has the amino acid sequences represented by SEQ ID NOs: 2 & 22. In another specific embodiment, the said antibody comprises the sequences encoded by the polynucleotide sequences represented by SEQ ID NOs: 25 & 21. In another specific embodiment, the said antibody has the amino acid sequences represented by SEQ ID NOs: 26 & 22.

A second aspect of the invention pertains to an antibody produced by the above method.

A third aspect of the invention pertains to pharmaceutical composition comprising the above antibody.

A fourth aspect of the invention pertains to the above antibody for use as a medicament.

A fifth aspect of the invention pertains to the above antibody for use in treating a disease associated with amyloid plaque formation, such as Alzheimer disease.

A sixth aspect of the invention pertains to a composition comprising an IgG antibody, wherein at least 80% of the said antibody comprises a complex, bi-antennary oligosaccharide attached each Fc domain of the said antibody, said oligosaccharide comprising two sialic acid residues, wherein the Fc domain comprises an amino sequence which differs from a native sequence human IgG Fc domain.

In a specific embodiment, the said sialic acid residues are linked to the antibody through an α-2,6-linkage.

In another specific embodiment, the antibody of the composition comprises an amino acid substitution at any one or more of amino acid positions 243, 264 and 265, such as a substitution selected from the group consisting of F243A, V264A, and D265A, and in particular a D265A substitution.

### DETAILED DESCRIPTION OF THE INVENTION

The invention relates to a method for producing an IgG antibody, wherein at least 80% of the said antibody comprises a complex, bi-antennary oligosaccharide, which contains two sialic acid residues, attached to each Fc domain of the antibody, said method comprising the steps of:

a) introducing a mutation in the said Fc domain of the said antibody, and
b) expressing the mutant antibody obtained in step a) in a cell line expressing a β galactosyltransferase and a sialyltransferase activity.

IgG immunoglobulins contain a single, N-linked glycan at Asn 297 in the CH2 domain on each of its two heavy chains, the structure of which is illustrated on FIG. 1. As used herein, the term “N-glycan” refers to an N-linked oligosaccharide, e.g., one that is attached by an asparagine-N-acetylglucosamine linkage to an asparagine residue of a polypeptide. N-glycans have a common pentasaccharide core of Man3GlcNAc2 (“Man” refers to mannose; GlcNAc refers to N-acetylglucosamine).

N-glycans differ with respect to the number and the nature of branches (antennae) comprising peripheral sugars (e.g., GlcNAc, galactose, fucose, and sialic acid) that are attached to the Man3 core structure. N-glycans are classified according to their branched constituents (e.g., high mannose, complex or hybrid). A “complex, bi-antennary” type N-glycan typically has at least one GlcNAc attached to the 1,3 mannose branch and at least one GlcNAc attached to the 1,6 mannose branch of the trimannose core. Complex bi-antennary N-glycans may also have intrachain substitutions comprising “bisecting” GlcNAc and core fucose (“Fuc”). A “bisecting GlcNAc” is a GlcNAc residue attached to the β-1,4-mannose of the mature core carbohydrate structure.

Complex bi-antennary N-glycans may also have galactose (“Gal”) residues that are optionally modified with sialic acid. Sialic acid addition to the oligosaccharide chain is catalyzed by a sialyltransferase, but requires previous attachment of one or more galactose residues by a galactosyltransferase to terminal N-acetylglucosamines. “Sialic acids” according to the invention encompass both 5-N-acetylneuraminic acid (NeuNAc) and 5-glycolylneuraminic acid (NeuNGc).

A secreted IgG is thus a heterogeneous mixture of glycoforms exhibiting variable addition of the sugar residues fucose, galactose, sialic acid, and bisecting N-acetylglucosamine.

The sialic acid residues can be linked to the galactose residues, and thus to the antibody, via either an α-2,3- or α-2,6-linkage. It has been shown that antibodies with α-2,6 sialylated N-glycan in the Fc domain have anti-inflammatory activity (Kaneko et al., Science, 313: 670-673, 2006; Jefferis, Nature Biotechnol., 24(10): 1230-1231, 2006; Anthony et al., Proc Natl Acad Sci U.S.A., 105: 19571-19578, 2008; Anthony et al., Science, 320: 373-376, 2008). In one embodiment of the invention, the two sialic acid residues are attached to the antibody via an α-2,6-linkage.

The term “antibody” is used herein in the broadest sense and specifically covers monoclonal antibodies (including full length monoclonal antibodies) of any isotype such as IgG, IgM, IgA, IgD, and IgE, polyclonal antibodies, multispecific antibodies, chimeric antibodies, and antibody fragments. An antibody reactive with a specific antigen can be generated by recombinant methods such as selection of libraries of recombinant antibodies in phage or similar vectors, or by immunizing an animal with the antigen or an antigen-encoding nucleic acid.

A “polyclonal antibody” is an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal.

A “monoclonal antibody”, as used herein, is an antibody obtained from a population of substantially homogeneous antibodies, i.e. the antibodies forming this population are essentially identical except for possible naturally occurring mutations which might be present in minor amounts. These antibodies are directed against a single epitope and are therefore highly specific.

An “epitope” is the site on the antigen to which an antibody binds. It can be formed by contiguous residues or by non-contiguous residues brought into close proximity by the folding of an antigenic protein. Epitopes formed by contiguous amino acids are typically retained on exposure to denaturing solvents, whereas epitopes formed by non-contiguous amino acids are typically lost under said exposure.

Preferably, the antibody of the invention is a monoclonal antibody.

A typical antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus (see Kabat et al., Sequences of Proteins of Immunological Interest, 5th edition, National Institute of Health, Bethesda, Md., 1991). The more highly conserved portions of the variable regions are called the “framework regions”.

As used herein, “VH” refers to the variable region of an immunoglobulin heavy chain of an antibody, including the heavy chain of an Fv, scFv, dsFv, Fab, Fab′, or F(ab′)2 fragment. Reference to “VL” refers to the variable region of the immunoglobulin light chain of an antibody, including the light chain of an Fv, scFv, dsFv, Fab, Fab′, or F(ab′)2 fragment.

Antibody constant domains are not involved directly in binding an antibody to an antigen, but exhibit various effector functions. The heavy chain constant regions that correspond to the different classes of immunoglobulins are called α, δ, ε, γ, and μ, respectively. Depending on the amino acid sequence of the constant region of their heavy chains, antibodies or immunoglobulins can be assigned to different classes, i.e., IgA, IgD, IgE, IgG, and IgM, and several of these may be further divided into subclasses (isotypes), e.g., IgG1, IgG2, IgG3, and IgG4; IgA1 and IgA2 (see, W. E. Paul, ed., 1993, Fundamental Immunology, Raven Press, New York, N.Y.).

Papain digestion of antibodies produces two identical antigen binding fragments, called Fab fragments, each with a single antigen binding site, and a residual “Fc” fragment. The crystal structure of the human IgG Fc domain has been determined (Deisenhofer, Biochemistry, 20, 2361-2370, 1981). As used in the specification and claims, “immunoglobulin Fc domain or Fc” means the carboxyl-terminal portion of the immunoglobulin heavy chain constant region. A “native sequence Fc domain”, as used herein, comprises an amino acid sequence identical to the amino acid sequence of a Fc domain found in nature. Native sequence human Fc domains include a native sequence human IgG1 Fc domain (non-A and A allotypes); native sequence human IgG2 Fc domain; native sequence human IgG3 Fc domain; and native sequence human IgG4 Fc domain as well as naturally occurring variants thereof.

Although the boundaries of the Fc domain of an immunoglobulin heavy chain might vary, the human IgG heavy chain Fc domain is usually defined to stretch from an amino acid residue at position Cys226 or Pro230 in the hinge region, to the carboxyl-terminus thereof containing the CH2 and CH3 domain of the heavy chain. Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

The term “hinge region” is generally defined as stretching from Glu216 to Pro230 of human IgG1 (Burton, Mol Immunol, 22: 161-206, 1985). Hinge regions of other IgG isotypes may be aligned with the IgG1 sequence by placing the first and last cysteine residues forming inter-heavy chain S—S bonds in the same positions. The “CH2 domain” of a human IgG Fc portion (also referred to as “Cγ2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton, Mol Immunol, 22: 161-206, 1985). The “CH3 domain” comprises the stretch of residues C-terminal to a CH2 domain in an Fc portion (i.e., from about amino acid residue 341 to about amino acid residue 447 of an IgG).

The Fc domains are central in determining the biological functions of the immunoglobulin and these biological functions are termed “effector functions”. These Fc domain-mediated activities are mediated via immunological effector cells, such as killer cells, natural killer cells, and activated macrophages, or various complement components. These effector functions involve activation of receptors on the surface of said effector cells, through the binding of the Fc domain of an antibody to the said receptor or to complement component(s). The antibody-dependent cellular cytotoxicity (ADCC) and complement-dependent cytotoxicity (CDC) activities involve the binding of the Fc domain to Fc-receptors such as FcγRI, FcγRII, FcγRIII of the effector cells or complement components such as C1q. Of the various human immunoglobulin classes, human IgG1 and IgG3 mediate ADCC more effectively than IgG2 and IgG4.

The antibody according to the invention comprises a mutation in the Fc domain. Advantageously, an Fc domain carrying the said mutation comprises more sialic acid residues than a native sequence Fc domain. Preferably, the said mutation affects an amino acid selected from the group consisting of F243, V264, and D265. More preferably, the said amino acid is substituted by an amino acid selected from the group consisting of alanine (A), glycine (G), leucine, (L), and lysine (K). Even more preferably, the said mutation is selected from the group consisting of F243A, V264A, D265A, D265G, D265L, and D265K. Still more preferably, the said mutation is selected from the group consisting of D265A, D265G, D265L, and D265K. Even more preferably, the said mutation is selected from the group consisting of D265A, D265K, and D265L.

The above amino acid positions correspond to the position given in the EU numbering as set forth in Kabat et al. (Sequences of Proteins of Immunological Interest, 5th edition, National Institute of Health, Bethesda, Md., 1991). The EU numbering has been used throughout the detailed description of the invention and throughout the claims. However, in the examples, the amino acid position is sometimes provided by reference to its location on the sequence of the murine 13C3 antibody or of the humanized 13C3 antibody. While the positions of the mutations are immediately apparent to the skilled in the art in view of the specification as a whole, the table below and FIG. 27 are provided for the sake of convenience.

| Position according | | |
|---|---|---|
| to the EU | Position on the murine | Position on the humanized |
| numbering | antiAbeta_13C3 mAb | antiAbeta_13C3 mAb |
| D265 | D257 | D260 |
| F243 | F235 | F238 |
| V264 | V256 | V259 |

In the frame of the present invention, the Fc domain may for example be a human IgG1 Fc domain (e.g. of SEQ ID NO: 57), a human IgG2 Fc domain (e.g. of SEQ ID NO: 58), a human IgG3 domain (see e.g. Lund et al., J. Immunol., 157: 4963-4969, 1996), a human IgG4 Fc domain (e.g. of SEQ ID NO: 59 or of SEQ ID NO: 60), a murine IgG1 Fc domain (e.g. of SEQ ID NO: 61), a murine IgG2a Fc domain (e.g. of SEQ ID NO: 62), or a murine IgG3 Fc domain (e.g. of SEQ ID NO: 63). It may correspond to a naturally-occurring Fc domain, or to a Fc domain in which mutations have been introduced by genetic engineering to enhance or reduce effector function of the antibody, and/or to enhance the half-life of the antibody. Such mutations are well-known to the skilled in the art.

In some embodiments, the method of the invention will comprise a preliminary step of introducing a mutation in the Fc domain of the antibody to be expressed. This can be performed using any suitable method known to the skilled person, e.g., oligonucleotide-mediated site-directed mutagenesis, cassette mutagenesis, error-prone PCR, DNA shuffling, or mutator strains of E. coli (Vaughan et al., Nature Biotech, 16: 535-539, 1998; Adey et al., 1996, Chapter 16, pp. 277-291, in “Phage Display of Peptides and Proteins”, Eds. Kay, et al., Academic Press).

In one embodiment, the antibody produced in the method of the invention is a humanized antibody.

As used herein, the term “humanized antibody” refers to a chimeric antibody which contains minimal sequence derived from non-human immunoglobulin. A “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass.

The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Strategies and methods for the resurfacing of antibodies, and other methods for reducing immunogenicity of antibodies within a different host, are disclosed in U.S. Pat. No. 5,639,641, which is hereby incorporated in its entirety by reference. Briefly, in a specific method, (1) position alignments of a pool of antibody heavy and light chain variable regions is generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 A of any atom of any residue of the complementarity-determining regions of the rodent antibody; and (5) the humanized rodent antibody having binding specificity is produced.

Another method of humanization of antibodies, based on the identification of flexible residues, has been described in PCT application WO 2009/032661. Said method comprises the following steps: (1) building an identity model of the parent monoclonal antibody and running a molecular dynamics simulation; (2) analyzing the flexible residues and identification of the most flexible residues of a non-human antibody molecule, as well as identifying residues or motifs likely to be a source of heterogeneity or of degradation reaction; (3) identifying a human antibody which displays the most similar ensemble of recognition areas as the parent antibody; (4) determining the flexible residues to be mutated, residues or motifs likely to be a source of heterogeneity and degradation are also mutated; and (5) checking for the presence of known T cell or B cell epitopes. The flexible residues can be found using an molecular dynamics calculation using an implicit solvent model, which accounts for the interaction of the water solvent with the protein atoms over the period of time of the simulation.

Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Mol Immunol, 28(4/5): 489-498; Studnicka G. M. et al., 1994, Protein Engineering 7(6): 805-814; Roguska M. A. et al., 1994, Proc. Natl. Acad. Sci. U.S.A., 91: 969-973), and chain shuffling (U.S. Pat. No. 5,565,332).

In one aspect, the antibody of the invention is a humanized antibody of the IgG isotype which specifically binds to the protofibrillar form of peptide A-β, i.e. a high-molecular weight peptide. More preferably, the antibody of the invention binds to a peptide A-β having a molecular weight superior or equal to 200, 300, 400 or 500 kDa.

The present invention also relates to a humanized antibody with reduced effector functions, which permits a diminution of adverse effects, such as microhaemorrhage. In one embodiment, the antibody of the invention does not have any effector function. In another embodiment, the antibody of the invention comprises an IgG4 Fc domain. In a yet further embodiment, the IgG4 Fc domain of the antibody of the invention contains one or more mutations which diminish the production of half-molecules. In another further embodiment, the Fc domain of the said antibody carries at least one mutation which leads to a reduction of the said antibody's effector functions.

Preferably, the antibody of the invention is a humanized antibody having at least one CDR coded by a polynucleotide having a sequence identical to a sequence selected from SEQ ID NOs: 9, 11, 13, 15, 17 and 19, or having a sequence differing from one of the said sequences by 1, 2, 3, 4, or 5 nucleotides.

The present invention also relates to a humanized antibody which has at least one CDR displaying a sequence identical to one sequence selected from SEQ ID NOs: 10, 12, 14, 16, 18, and 20. In another aspect, the antibody of the invention has at least one CDR which differs from the said sequences by 1 or 2 amino acid residues, while retaining its binding specificity.

In one embodiment, the antibody of the invention comprises 6 CDRs encoded by the nucleotide sequences SEQ ID NOs: 9, 11, 13, 15, 17, and 19, or by variants thereof differing only by 1, 2, 3, 4, or 5 nucleotides from the said sequences. In another embodiment, the antibody of the invention comprises 6 CDRs having sequences identical to the sequences represented by SEQ ID NOs: 10, 12, 14, 16, 18, and 20.

In another embodiment, the antibody of the invention comprises 6 CDRs encoded by the nucleotide sequences SEQ ID NOs: 9, 11, 13, 31, 17, and 19, or by variants thereof differing only by 1, 2, 3, 4, or 5 nucleotides from the said sequences. In still another embodiment, the antibody of the invention comprises 6 CDRs having sequences identical to the sequences represented by SEQ ID NOs: 10, 12, 14, 32, 18, and 20.

In yet another embodiment, the antibody of the invention comprises 6 CDRs encoded by the nucleotide sequences SEQ ID NOs: 9, 11, 29, 31, 17, and 19, or by variants thereof differing only by 1, 2, 3, 4, or 5 nucleotides from the said sequences. In another embodiment, the antibody of the invention comprises 6 CDRs having sequences identical to the sequences represented by SEQ ID NOs: 10, 12, 30, 32, 18, and 20.

In another aspect, the invention relates to an antibody which comprises a VH encoded by a polynucleotide sequence displaying at least 80, 85, 90, 95, or 99% identity with the sequence represented by SEQ ID NO: 5 or the sequence represented by SEQ ID NO: 27. In one embodiment, the sequence coding the VH of the antibody of the invention is selected between SEQ ID NO: 5 and SEQ ID NO: 27. In another embodiment, the VH of the antibody of the invention has a sequence having at least 80, 85, 90, 95, or 99% identity with the sequence represented by SEQ ID NO: 6 or the sequence represented by SEQ ID NO: 28. In a further embodiment, the sequence of the VH of the antibody of the invention is represented by SEQ ID NO: 6 or SEQ ID NO: 28.

In another aspect, the invention provides an antibody which VL is encoded by a polynucleotide sequence displaying at least 80, 85, 90, 95, or 99% identity with the sequence represented by SEQ ID NO: 7 or the sequence represented by SEQ ID NO: 23. Preferably, the VL of the antibody of the invention is encoded by a polynucleotide sequence represented by SEQ ID NO: 7 or SEQ ID NO: 23. In another embodiment, the VL of the antibody of the invention has a sequence having at least 80, 85, 90, 95, or 99% identity with the sequence represented by SEQ ID NO: 8 or the sequence represented by SEQ ID NO: 24. In a further embodiment, the sequence of the VL of the antibody of the invention is represented by SEQ ID NO: 8 or SEQ ID NO: 24.

In one embodiment, the invention provides an antibody which comprises the sequences encoded by the polynucleotide sequences SEQ ID NOs: 5 & 7. In a further embodiment, the invention comprises the amino acid sequences represented by SEQ ID NOs: 6 & 8.

In another embodiment, the invention provides an antibody which comprises the sequences encoded by the polynucleotide sequences SEQ ID NOs: 5 & 23. In a further embodiment, the invention comprises the amino acid sequences represented by SEQ ID NOs: 6 & 24.

In yet another embodiment, the invention provides an antibody which comprises the sequences encoded by the polynucleotide sequences SEQ ID NOs: 27 & 23. In a further embodiment, the invention comprises the amino acid sequences represented by SEQ ID NOs: 28 & 24.

The present invention also relates to an antibody comprising a heavy chain encoded by a polynucleotide sequence having at least 80%, 85%, 90%, 95%, or 99% identity with a sequence represented by SEQ ID NO: 1 or SEQ ID NO: 25. The present invention also relates to an antibody comprising a heavy chain having an amino acid sequence with at least 80%, 85%, 90%, 95%, or 99% identity with a sequence represented by SEQ ID NO: 2 or SEQ ID NO: 26.

In another aspect, the present invention provides an antibody comprising a light chain encoded by a polynucleotide sequence having at least 80%, 85%, 90%, 95%, or 99% identity with a sequence represented by SEQ ID NO: 3 or SEQ ID NO: 21. The present invention also relates to an antibody comprising a light chain having an amino acid sequence with at least 80%, 85%, 90%, 95%, or 99% identity with a sequence represented by SEQ ID NO: 4 or SEQ ID NO: 22.

Another aspect of the invention relates to an antibody which comprises the sequences encoded by the polynucleotide sequences represented by SEQ ID NOs: 1 & 3. Preferably, the antibody of the invention has the amino acid sequences represented by SEQ ID NOs: 2 & 4.

Another aspect of the invention relates to an antibody which comprises the sequences encoded by the polynucleotide sequences represented by SEQ ID NOs: 1 & 21. Preferably, the antibody of the invention has the amino acid sequences represented by SEQ ID NOs: 2 & 22.

Another aspect of the invention relates to an antibody which comprises the sequences encoded by the polynucleotide sequences represented by SEQ ID NOs: 25 & 21. Preferably, the antibody of the invention has the amino acid sequences represented by SEQ ID NOs: 26 & 22.

The sequences encoding or constituting the antibodies of the invention are displayed in Table 1.

TABLE 1

| | | | Name of the |
|---|---|---|---|
| SEQ ID Nos | Nature | Domain | antibody |
| 1 | DNA | HC | humanized |
| 2 | Protein | HC | 13C3 |
| 3 | DNA | LC | |
| 4 | Protein | LC | |
| 5 | DNA | VH | |
| 6 | Protein | VH | |
| 7 | DNA | VL | |
| 8 | Protein | VL | |
| 9 | DNA | CDR | |
| 10 | Protein | CDR | |
| 11 | DNA | CDR | |
| 12 | Protein | CDR | |
| 13 | DNA | CDR | |
| 14 | Protein | CDR | |
| 15 | DNA | CDR | |
| 16 | Protein | CDR | |
| 17 | DNA | CDR | |
| 18 | Protein | CDR | |
| 19 | DNA | CDR | |
| 20 | Protein | CDR | |
| 21 | DNA | LC | |
| 22 | Protein | LC | |
| 23 | DNA | VL | |
| 24 | Protein | VL | |
| 25 | DNA | HC | |
| 26 | Protein | HC | |
| 27 | DNA | VH | |
| 28 | Protein | VH | |
| 29 | DNA | CDR | |
| 30 | Protein | CDR | |
| 31 | DNA | CDR | |
| 32 | Protein | CDR | |
| 33 | DNA | SIAT1 | Not applicable |
| 34 | Protein | SIAT1 | |
| 35 | DNA | B4GT1 | |
| 36 | Protein | B4GT1 | |
| 37 | DNA | HC | murine |
| 38 | Protein | HC | 13C3 |
| 39 | DNA | LC | |
| 40 | Protein | LC | |
| 41 | DNA | HC | murine |
| 42 | Protein | HC | 13C3 F235 A |
| 43 | DNA | HC | murine |
| 44 | Protein | HC | 13C3 V256A |
| 45 | DNA | HC | murine |
| 46 | Protein | HC | 13C3 D257A |
| 47 | DNA | HC | humanized |
| 48 | Protein | HC | 13C3 D260A |
| 49 | DNA | HC | humanized |
| 50 | Protein | HC | 13C3 D260G |
| 51 | DNA | HC | humanized |
| 52 | Protein | HC | 13C3 D260L |
| 53 | DNA | HC | humanized |
| 54 | Protein | HC | 13C3 D260K |
| 55 | DNA | HC | humanized |
| 56 | Protein | HC | 13C3 D260S |

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e. a portion of a complete polynucleotide sequence) that is similar between two polynucleotides, and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e. gaps) of 20 percent or less compared to the reference sequence (which does not comprise additions or deletions) for optimal alignment of the two sequences.

To determine the percent identity of two amino acids sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position. The percent identity between the two sequences is a function of the number of identical positions shared by the sequences. Hence % identity=number of identical positions/total number of overlapping positions×100.

In this comparison the sequences can be the same length or can be different in length. Optimal alignment of sequences for determining a comparison window may be conducted by the local identity algorithm of Smith and Waterman (J. Theor. Biol., 91(2): 370-380, 1981), by the identity alignment algorithm of Needleman and Wunsch (J. Mol. Biol, 48(3): 443-453, 1972), by the search for similarity via the method of Pearson and Lipman (Proc. Natl. Acad. Sci. U.S.A., 85(5): 2444-2448, 1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection. The best alignment (i.e. resulting in the highest percentage of identity over the comparison window) generated by the various methods is selected.

The term “sequence identity” means that two polynucleotide or polypeptide sequences are identical (i.e. on a nucleotide by nucleotide or an amino acid by amino acid basis) over the window of comparison. The term “percentage of sequence identity” is calculated by comparing two optimally aligned sequences over the window of comparison, determining the number of positions at which the identical nucleic acid base (e.g. A, T, C, G, U, or I) occurs in both sequences to yield the number of matched positions, dividing the number of matched positions by the total number of positions in the window of comparison (i.e. the window size) and multiplying the result by 100 to yield the percentage of sequence identity. The same process can be applied to polypeptide sequences. The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

According to the invention, the sialic acid residue(s) are added onto the antibody of the invention during expression by the host cell. The host cell according to the invention overexpresses a β galactosyltransferase and a sialyltransferase.

By “β galactosyltransferase”, it is herein referred to an enzyme which is capable of covalently linking a galactose residue to an N-acetylglucosamine residue on an N-glycan of a glycoprotein. Preferentially, the said enzyme is a β-1,4-galactosyltransferase (EC=2.4.1.-). For example, the said enzyme is the β-1,4-galactosyltransferase, known as β-1,4-galactosyltransferase 1 (Genbank accession number: NP_{—}001488.2), encoded by the gene B4GALT1 (Genbank accession number: NM_{—}0014973). More preferentially, the β-1,4-galactosyltransferase has the amino acid sequence represented by SEQ ID NO: 36, and is encoded by the polynucleotide sequence represented by SEQ ID NO: 35.

A “sialyltransferase” according to the invention is an enzyme capable of linking a sialyl acid residue to a galactose residue on an N-glycan of a glycoprotein. Suitable non-limiting examples of sialyltransferase enzymes useful in the claimed methods are ST3Gal III, which is also referred to as α-2,3-sialyltransferase (EC 2.4.99.6), and α-2,6-sialyltransferase (EC 2.4.99.1).

Alpha-2,3-sialyltransferase catalyzes the transfer of a sialic acid residue to the Gal of a Gal-β-1,3GlcNAc or Gal-β-1,4GlcNAc glycoside (see, e.g., Wen et al., J. Biol. Chem. 267: 21011-21019, 1992) and is responsible for sialylation of N-linked oligosaccharides in glycopeptides. The sialic acid residue is linked to the galactose with the formation of an α-linkage between the two saccharides. Bonding (linkage) between the saccharides is between the 2-position of the sialic acid residue and the 3-position of the galactose residue. This particular enzyme can be isolated from rat liver (Weinstein et al., J. Biol. Chem., 257: 13845-13853, 1982); the human cDNA (Sasaki et al., J. Biol. Chem., 268: 22782-22787, 1993; Kitagawa & Paulson, J. Biol. Chem., 269: 1394-1401, 1994) and genomic (Kitagawa et al., J. Biol. Chem., 271: 931-938, 1996) DNA sequences are known, facilitating production of this enzyme by recombinant expression.

Activity of α-2,6-sialyltransferase results in α-2,6-sialylated oligosaccharides, including α-2,6-sialylated galactose. The name “α-2,6-sialyltransferase” refers to the family of sialyltransferases attaching sialic acid to the sixth atom of the acceptor polysaccharide. Different forms of α-2,6-sialyltransferase can be isolated from different tissues. For example, one specific form of this enzyme, ST6Gal II, can be isolated from brain and fetal tissues (Krzewinski-Recchi et al., Eur. J. Biochem., 270: 950-961, 2003). Preferentially, the α-2,6-sialyltransferase is a β galactoside α-2,6-sialyltransferase (Genbank accession number: NP_{—}003023.1), encoded by the SIAT1 gene (Genbank accession number: NM_{—}003032). More preferentially, the α-2,6-sialyltransferase has the amino acid sequence represented by SEQ ID NO: 34, and is encoded by the polynucleotide sequence represented by SEQ ID NO: 33.

The method of the invention thus allows for the obtention of extensively sialylated antibodies, wherein most of the covalent bonds between galactose and sialic acid are either in α-2,3 or α-2,6, depending on the enzyme used. It is especially advantageous to use a host cell which overexpresses a β-1,4-galactosyltransferase and an α-2,6-sialyltransferase. The oligosaccharide carried by the resulting antibodies thus comprises mostly sialic acid residues bound to galactose residues via an α-2,6 linkage.

A desired host cell may thus be transfected in order to transiently or stably express one of these enzymes or both. Therefore, in a specific embodiment of the method according to the invention, the cell line expressing a β-galactosyltransferase and a sialyltransferase activity is a cell line that has been stably transfected with one or two vectors encoding beta-galactosyltransferase and sialyltransferase (e.g. a first vector expressing the beta-galactosyltransferase and a second vector expressing the sialyltransferase, or one vector expressing both enzymes). Preferably a α-2,6-sialyltransferase and/or a β-1,4-galactosyltransferase of rodent, e.g. mouse or rat, or human origin is used for addition of sialic acid residues to the expressed antibody. Most preferably, the α-2,6-sialyltransferase and/or the β-1,4galactosyltransferase used in the method of the invention are the human enzymes. In a particularly advantageous embodiment of the invention, the host cell overexpresses both a human β-1,4-galactosyltransferase and a human α-2,6sialyltransferase.

The nucleic acids encoding the β galactosyltransferase and sialyltransferase may be introduced into the host cell by any method known to a person of ordinary skills in the art (see, for example, the techniques described in Sambrook et al., 1990, Molecular Cloning, A Laboratory Manual, 2nd Ed., Cold Spring Harbor Laboratory, Cold Spring Harbor, N.Y. and Ausubel et al., eds., 1998, Current Protocols in Molecular Biology, John Wiley & Sons, NY). These methods include, without limitation, transfections (e;g. calcium phosphate transfection), membrane fusion transfer using for example liposome, viral transfer (with e.g. adenoviral vector) and microinjection or electroporation.

According to the invention, a variety of expression systems may be used to express the IgG antibody of the invention. In one aspect, such expression systems represent vehicles by which the coding sequences of interest may be produced and subsequently purified, but also represent cells which may, when transiently transfected with the appropriate nucleotide coding sequences, express an IgG antibody of the invention in situ.

The invention provides vectors comprising the polynucleotides of the invention. In one embodiment, the vector contains a polynucleotide encoding a heavy chain of an IgG antibody of the invention, i.e. an antibody which carries a mutation in the Fc domain. In another embodiment, said polynucleotide encodes the light chain of an IgG antibody of the invention. The invention also provides vectors comprising polynucleotide molecules encoding fusion proteins, modified antibodies, antibody fragments, and probes thereof.

In order to express the heavy and/or light chain of the an IgG antibody of the invention, the polynucleotides encoding said heavy and/or light chains are inserted into expression vectors such that the genes are operatively linked to transcriptional and translational sequences.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome.

Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector. However, the invention is intended to include such forms of expression vectors, such as bacterial plasmids, YACs, cosmids, retrovirus, EBV-derived episomes, and all the other vectors that the skilled man will know to be convenient for ensuring the expression of the heavy and/or light chains of the antibodies of the invention. The skilled man will realize that the polynucleotides encoding the heavy and the light chains can be cloned into different vectors or in the same vector. In one embodiment, said polynucleotides are cloned into two vectors.

Polynucleotides of the invention and vectors comprising these molecules can be used for the transformation of a suitable host cell. The term “host cell”, as used herein, is intended to refer to a cell into which a recombinant expression vector has been introduced in order to express the IgG antibody of the invention. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

Transformation can be performed by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei.

The host cell may be co-transfected with two or more expression vectors, including the vector expressing the protein of the invention. For example, a host cell can be transfected with a first vector encoding an IgG antibody, as described above, and a second vector encoding a glycosyltransferase polypeptide. Alternatively, the host cell can be transformed with a first vector encoding an antibody of the invention, a second vector encoding a glycosyltransferase, as described above, and a third vector encoding another glycosyltransferase. Mammalian cells are commonly used for the expression of a recombinant therapeutic immunoglobulins, especially for the expression of whole recombinant IgG antibodies. For example, mammalian cells such as HEK293 or CHO cells, in conjunction with a vector, containing the expression signal such as one carrying the major intermediate early gene promoter element from human cytomegalovirus, are an effective system for expressing the IgG antibody of the invention (Foecking et al., 1986, Gene 45:101; Cockett et al., 1990, Bio/Technology 8: 2).

In addition, a host cell is chosen which modulates the expression of the inserted sequences, or modifies and processes the gene product in the specific fashion desired. Such modifications (e.g., glycosylation) and processing of protein products may be important for the function of the protein. Different host cells have features and specific mechanisms for the post-translational processing and modification of proteins and gene products. Appropriate cell lines or host systems are chosen to ensure the correct modification and processing of the expressed antibody of interest. Hence, eukaryotic host cells (and in particular mammalian host cells) which possess the cellular machinery for proper processing of the primary transcript, glycosylation of the gene product may be used. Such mammalian host cells include, but are not limited to, Chinese hamster cells (e.g. CHO cells), monkey cells (e.g. COS cells), human cells (e.g. HEK293 cells), baby hamster cells (e.g. BHK cells), NS/0, Y2/0, 3T3 or myeloma cells (all these cell lines are available from public depositories such as the Collection Nationale des Cultures de Microorganismes, Paris, France, or at the American Type Culture Collection, Manassas, Va., U.S.A.). Alternatively, the yeast cell may be a yeast cell that has been engineered so that the glycosylation (and in particular N-glucosylation) mechanisms are similar or identical to those taking place in a mammalian cell.

For long-term, high-yield production of recombinant proteins, stable expression is preferred. In one embodiment of the invention, cell lines which stably express the antibody may be engineered. Thus, in a specific embodiment of the method according to the invention, the cell line expressing a β-galactosyltransferase and a sialyltransferase activity has been stably transfected with one or two vectors encoding the antibody (e.g. a first vector expression the light chain and a second vector expressing the heavy chain, or one vector expressing both chains). Rather than using expression vectors which contain viral origins of replication, host cells are transformed with DNA under the control of the appropriate expression regulatory elements, including promoters, enhancers, transcription terminators, polyadenylation sites, and other appropriate sequences known to the person skilled in art, and a selectable marker. Following the introduction of the foreign DNA, engineered cells may be allowed to grow for one to two days in an enriched media, and then are moved to a selective media. The selectable marker on the recombinant plasmid confers resistance to the selection and allows cells to stably integrate the plasmid into a chromosome and be expanded into a cell line. Other methods for constructing stable cell lines are known in the art. In particular, methods for site-specific integration have been developed. According to these methods, the transformed DNA under the control of the appropriate expression regulatory elements, including promoters, enhancers, transcription terminators, polyadenylation sites, and other appropriate sequences is integrated in the host cell genome at a specific target site which has previously been cleaved (Moele et al., Proc. Natl. Acad. Sci. U.S.A., 104(9): 3055-3060; U.S. Pat. No. 5,792,632; U.S. Pat. No. 5,830,729; U.S. Pat. No. 6,238,924; WO 2009/054985; WO 03/025183; WO 2004/067753).

A number of selection systems may be used according to the invention, including but not limited to the Herpes simplex virus thymidine kinase (Wigler et al., Cell 11:223, 1977), hypoxanthine-guanine phosphoribosyltransferase (Szybalska et al., Proc Natl Acad Sci USA 48: 202, 1992), glutamate synthase selection in the presence of methionine sulfoximide (Adv Drug Del Rev, 58: 671, 2006, and website or literature of Lonza Group Ltd.) and adenine phosphoribosyltransferase (Lowy et al., Cell 22: 817, 1980) genes in tk, hgprt or aprt cells, respectively. Also, antimetabolite resistance can be used as the basis of selection for the following genes: dhfr, which confers resistance to methotrexate (Wigler et al., Proc Natl Acad Sci USA 77: 357, 1980); gpt, which confers resistance to mycophenolic acid (Mulligan et al., Proc Natl Acad Sci USA 78: 2072, 1981); neo, which confers resistance to the aminoglycoside, G-418 (Wu et al., Biotherapy 3: 87, 1991); and hygro, which confers resistance to hygromycin (Santerre et al., Gene 30: 147, 1984). Methods known in the art of recombinant DNA technology may be routinely applied to select the desired recombinant clone, and such methods are described, for example, in Ausubel et al., eds., Current Protocols in Molecular Biology, John Wiley & Sons (1993). The expression levels of an antibody can be increased by vector amplification. When a marker in the vector system expressing an antibody is amplifiable, an increase in the level of inhibitor present in the culture will increase the number of copies of the marker gene. Since the amplified region is associated with the gene encoding the IgG antibody of the invention, production of said antibody will also increase (Crouse et al., Mol Cell Biol 3: 257, 1983). Alternative methods of expressing the gene of the invention exist and are known to the person of skills in the art. For example, a modified zinc finger protein can be engineered that is capable of binding the expression regulatory elements upstream of the gene of the invention; expression of the said engineered zinc finger protein (ZFP) in the host cell of the invention leads to increases in protein production (see e.g. Reik et al., Biotechnol. Bioeng., 97(5): 1180-1189, 2006). Moreover, ZFN (Zinc Finger Nuclease) can stimulate the integration of a DNA into a predetermined genomic location, resulting in high-efficiency site-specific gene addition (Moehle et al, Proc Natl Acad Sci USA, 104: 3055, 2007).

The antibody of the invention may be prepared by growing a culture of the transformed host cells under culture conditions necessary to express the desired antibody. The resulting expressed antibody may then be purified from the culture medium or cell extracts. Soluble forms of the antibody of the invention can be recovered from the culture supernatant. It may then be purified by any method known in the art for purification of an immunoglobulin molecule, for example, by chromatography (e.g., ion exchange, affinity, particularly by Protein A affinity for Fc, and so on), centrifugation, differential solubility or by any other standard technique for the purification of proteins. Suitable methods of purification will be apparent to a person of ordinary skills in the art. The IgG antibody of the present invention can be further purified on the basis of its increased amount of sialic acid compared to unmodified and/or unpurified antibodies. Multiple methods exist to reach this objective. In one method, the source of unpurified polypeptides, such as, for example, the culture medium of the host cell of the invention is passed through the column having lectin, which is known to bind sialic acid. A person of the ordinary skill in the art will appreciate that different lectins display different affinities for α-2,6 versus α-2,3 linkages between galactose and sialic acid. Thus, selecting a specific lectin will allow enrichment of antibodies with the desired type of linkage between the sialic acid and the galactose. In one embodiment, the lectin is isolated from Sambucus nigra. A person of the ordinary skill in the art will appreciate that the Sambucus nigra agglutinin (SNA) is specific for sialic acids linked to galactose or N-acetylgalactosamine by α-2-6 linkages (Shibuya et al, J. Biol. Chem., 262: 1596-1601, 1987). In contrast, the Maakia amurensis (“MAA”) lectin is specific to sialic acid linked to galactose by α-2-3 linkages (Wang et al, J Biol. Chem., 263: 4576-4585, 1988).

To examine the extent of glycosylation on the polypeptides containing at least one IgG Fc domain, these polypeptides can be purified and analyzed in SDS-PAGE under reducing conditions. The glycosylation can be determined by reacting the isolated polypeptides with specific lectins, or, alternatively as would be appreciated by one of ordinary skill in the art, one can use HPLC followed by mass spectrometry to identify the glycoforms (Wormald et al., Biochem, 36(6): 1370-1380, 1997). Quantitative sialic acid identification (N-acetylneuraminic acid residues), carbohydrate composition analysis and quantitative oligosaccharide mapping of N-glycans in the IgG antibody can be performed essentially as described previously (Saddic et al., Methods Mol. Biol., 194: 23-36, 2002; Anumula et al., Glycobiology, 8:685-694, 1998).

The method of the invention thus allows the production of an antibody comprising a complex, bi-antennary oligosaccharide, which contains two sialic acid residues, attached to the Fc domain of the said antibody, with a high productivity. “High productivity” as used herein means that the said antibody can be produced at yields superior or equal to 25 mg/L, preferably 30 mg/L, more preferably 35 mg/L, still more preferably 40 mg/L, even more preferably 45 mg/L, or most preferably 50 mg/L or more.

The invention also relates to a purified, extensively-sialylated IgG antibody, which can be obtained by the above-described method. The said antibody is an antibody of the IgG isotype, comprising a complex, bi-antennary, extensively-sialylated N-glycan on each Fc domain, said antibody carrying a mutation in the Fc domain. Preferably, the antibody of the invention carries an oligosaccharide of the G2F form, i.e. each N-glycan of the said antibody comprises two galactose residues and one fucose. More preferably, the said N-glycan of the antibody of the invention comprises two sialic acid residues. Even more preferably, the sialic acid residues are linked to the galactose residues through α-2,6 bonds. Still more preferably, the sialic acid residues are both 5-N-acetylneuraminic acid residues (NeuNAc).

Preferably, the antibody of the invention is a humanized antibody which specifically binds to the protofibrillar form of peptide A-β and can thus be used for treating diseases associated with amyloid plaque formation. In particular, the humanized antibodies of the invention can be used for treating AD. More preferably, the said humanized antibody has reduced effector functions, and thus leads to reduced adverse effects. Because of its extensive sialylation, the said humanized antibody shows anti-inflammatory properties. The humanized antibody of the invention thus shows therapeutic efficacy combined with higher safety.

The inventors have shown for the first time that it is possible to obtain a composition of IgG antibodies, wherein a very high proportion of the said antibodies is extensively-sialylated (see e.g. Table 3). The invention thus also provides a composition comprising an IgG antibody of the invention, wherein at least 80%, preferably at least 85%, more preferably at least 90%, even more preferably at least 95%, still most preferably at least 97% or most preferably at least 99% of the said antibody is a purified, extensively-sialylated IgG antibody. The invention thus provides a composition comprising an IgG antibody, wherein at least 80%, preferably at least 85%, more preferably at least 90%, even more preferably at least 95%, still most preferably at least 97% or most preferably at least 99% of the said antibody comprises a complex, bi-antennary N-glycan attached each Fc domain of the said antibody, said oligosaccharide comprising two sialic acid residues, wherein the Fc domain of the said antibody comprises an amino acid sequence which differs from a native human IgG Fc domain sequence. Preferably, the antibody of the composition of the invention comprises an amino acid substitution at any one or more of amino acid positions 243, 264 and 265. More preferably, the said amino acid is substituted by an amino acid selected from the group consisting of alanine (A), glycine (G), leucine, (L) and lysine (K). Even more preferably, the substitutions are selected in the group comprising F243A, V264A, D265A, D265G, D265L, and D265K. Still more preferably, the said mutation is selected from the group consisting of D265A, D265G, D265L, and D265K. Most preferably, the said mutation is selected from the group consisting of D265A, D265K, and D265L.

Indeed, the inventors have advantageously shown that mutations at one of position F243, V264 and D265 leads to the obtention of antibodies species that exhibit a very homogeneous sialylation profile (see FIGS. 12B, C and D), said species being fully characterized and defined (see Table 3). In contrast to this, the absence of such mutations resulted in the production of a mixture of at least 12 different species containing non-sialylated or incompletely sialylated N-glycans (FIG. 12A).

It is important to note that not every mutation at position 265 leads to an increased sialylation. For example, a D265S substitution behaves like the wild-type in that respect, whereas a D265A, a D265G, a D265L, or a D265K mutation all lead to an enhanced proportion of disialylated antibody molecules, thus emphasizing the specificity of the mutants of the invention (see Example 7).

In a specific embodiment, the mutation is a mutation at position D265 (e.g. a D265L, D265K or D265A mutation). Indeed, the inventors have surprisingly found that a mutation at this position not only results in an extensively sialylated antibody, but also in an antibody that exhibits increased binding to its target (see Example 6 and FIG. 16B).

In another aspect, the antibody of the invention comprises a heavy chain which has a sequence selected from the group consisting of SEQ ID NOs: 48, 50, 52, and 54. Preferably, the heavy chain of the antibody of the invention has a sequence chosen between SEQ ID NO: 48, SEQ ID NO: 52, and SEQ ID NO: 54.

In another advantageous embodiment, the antibody of the composition of the invention carries an oligosaccharide of the G2F form, i.e. each N-glycan of the said antibody comprises two galactose residues and one fucose. Preferably, the sialic acid residues are linked to the galactose residues through α-2,6 bonds. More preferably, the sialic acid residues are both 5-N-acetylneuraminic acid residues (NeuNAc).

It was long known that the anti-inflammatory property is determined by the Fc portion of the IVIG. A mouse lectin, SIGN-R1 (Kang et al., Int. Immunol., 15(2): 177-186, 2003), expressed on the surface of splenic macrophages, is a receptor for α-2,6-sialylated Fc fragments, as is the human lectin, DC-SIGN expressed on human dendritic cells (Anthony et al., Proc. Natl. Acad. Sci. USA, 105(50): 19571-19578, 2008). The interaction of the α-2,6-sialyl acid residues with the said receptor is associated with the anti-inflammatory activity of the said immunoglobulins.

In an advantageous embodiment, the antibody composition of the invention binds SIGN-R1 or DC-SIGN, thus showing anti-inflammatory activity. Preferably, the humanized antibody composition of the invention binds SIGN-R1 or DC-SIGN with greater affinity than a composition wherein less than 5% of the antibody carries at least one disialylated N-glycan. By “SIGN-R1”, it is herein referred to the protein which is also designated “CD209 antigen-like protein A” and which has an amino acid sequence as in NP_{—}573501.1. By “DC-SIGN”, it is herein meant a protein with an amino acid sequence as in AAK20997. More preferably, the receptor bound by the humanized antibody composition of the invention is DC-SIGN.

The inventors have shown that, the antibodies produced according to the invention, and carrying in their Fc domain a D265A mutation show the highest affinity for SIGN-R1. Thus, the antibodies produced according to the invention and containing a mutation selected from the group consisting of D265A, D265G, D265K and D265L, would provided highest affinity to SIGN-R1. Even more preferentially, the antibody of the invention has a heavy chain which sequence is chosen between SEQ ID NO: 48, SEQ ID NO: 52, and SEQ ID NO: 54.

The invention thus also relates to the antibody of the invention as a medicament.

It is another object of the invention to provide a method of treating a disease associated with amyloid plaque formation, said method comprising the administration to a patient in need thereof of a humanized antibody of the IgG isotype, comprising a complex, bi-antennary, extensively-sialylated N-glycan on the Fc domain, said humanized antibody carrying a mutation in the Fc domain. The invention also relates to a humanized antibody of the IgG isotype for use in treating a disease associated with amyloid plaque formation, said humanized antibody comprising a complex, bi-antennary, extensively-sialylated N-glycan on the Fc domain, and said humanized antibody carrying a mutation in the Fc domain. The invention further relates to the use of a humanized antibody of the IgG isotype for the manufacture of a medicament for treating a disease associated with amyloid plaque formation, said humanized antibody comprising a complex, bi-antennary, extensively-sialylated N-glycan on the Fc domain, and said humanized antibody carrying a mutation in the Fc domain. In one embodiment, the disease associated with amyloid plaque formation is AD. In another embodiment, the sialic acid residues are linked to the galactose residues through α-2,6 bonds.

In another aspect, the invention relates to a pharmaceutical composition for the treatment of disease associated with amyloid plaque formation, in particular AD, said therapeutic composition comprising a therapeutically effective amount of a humanized antibody of the invention and a pharmaceutically acceptable carrier.

The pharmaceutical composition of the invention may contain, in addition to the antibody of the invention, various diluents, fillers, salts, buffers, stabilizers, solubilizers, and other materials well known in the art.

As used herein, “pharmaceutically acceptable carrier” includes any and all solvents, buffers, salt solutions, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of media and agents for pharmaceutically active substances is well known in the art. A typical pharmaceutical composition for intravenous infusion could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17th ed., Mack Publishing Company, Easton, Pa. (1985), and the 18th and 19th editions thereof, which are incorporated herein by reference.

The humanized antibody in the composition preferably is formulated in an effective amount. An “effective amount” refers to an amount effective, at dosages and for periods of time necessary, to achieve the desired result, such as prevention or treatment of amyloid plaque formation. A “therapeutically effective amount” means an amount sufficient to influence the therapeutic course of a particular disease state. A therapeutically effective amount is also one in which any toxic or detrimental effects of the agent are outweighed by the therapeutically beneficial effects.

For therapeutic applications, the humanized antibody of the invention is administered to a mammal, preferably a human, in a pharmaceutically acceptable dosage form such as those discussed above, including those that may be administered to a human intravenously as a bolus or by continuous infusion over a period of time, by intramuscular, intraperitoneal, intracerebrospinal, subcutaneous, intraarticular, intrasynovial, intrathecal, oral, topical, or inhalation routes.

Dosage regimens may be adjusted to provide the optimum response. For example, a single bolus may be administered, several divided doses may be administered over time, or the dose may be proportionally reduced or increased. The compositions of the invention can be administered to a subject to effect cell growth activity in a subject. As used herein, the term “subject” is intended to include living organisms in which apoptosis can be induced, and specifically includes mammals, such as rabbits, dogs, cats, mice, rats, monkey transgenic species thereof, and preferably humans.

The examples that follow are merely exemplary of the scope of this invention and content of this disclosure. One skilled in the art can devise and construct numerous modifications to the examples listed below without departing from the scope of this invention.

### BRIEF DESCRIPTION OF THE FIGURES

FIG. 1. Structures of two N-glycans, G0F and G2F+2 NeuNAc. Monosaccharide composition of N-glycans is presented using standard pictograms for each monosaccharide, i.e. fucose, N-acetylglucosamine, mannose, galactose and N-acetylneuraminic acid.

FIG. 2. Maps of the expression plasmids pXL4555 (FIG. 2A) and pXL4551 (FIG. 2B) coding for SIAT1 and B4GT1 respectively.

FIG. 3. Nucleic acid sequence (SEQ ID No.33) (FIG. 3A) and amino acid sequence (SEQ ID No. 34) (FIG. 3B) of SIAT1 for expression from expression plasmid pXL4555.

FIGS. 4A and 4B. Nucleic acid sequence (SEQ ID No.35) (FIG. 4A) and amino acid sequence (SEQ ID No. 36) (FIG. 4B) of B4GT1 for expression from expression plasmid pXL4551.

FIG. 5. Maps of expression plasmids pXL4808 coding for the light chain (LC) of antiAbeta_{—}13C13 mAb (FIG. 5A); pXL4792 coding for the heavy chain (HC) of antiAbeta_{—}13C13 mAb (FIG. 5B); pXL5105 coding for the modified HC of AntiAbeta_{—}13C3_D257A (FIG. 5C); pXL5111 coding for the modified HC of AntiAbeta_{—}13C3_F235A mAb (FIG. 5D); and pXL5132 coding for the modified HC of AntiAbeta_{—}13C3_V256A mAb (FIG. 5E).

FIG. 6. Nucleic acid sequence (SEQ ID No.39) (FIG. 6A) and amino acid sequence (SEQ ID No. 40) (FIG. 6B) of the LC antiAbeta_{—}13C13 mAb for expression from expression plasmid pXL4808.

FIG. 7. Nucleic acid sequence (SEQ ID No.37) (FIG. 7A) and amino acid sequence (SEQ ID No. 38) (FIG. 7B) of the HC antiAbeta_{—}13C13 mAb for expression from expression plasmid pXL4792.

FIG. 8. Nucleic acid sequence (SEQ ID No. 45) (FIG. 8A) and amino acid sequence (SEQ ID No. 46) (FIG. 8B) of the HC antiAbeta_{—}13C13_D257A mAb for expression from expression plasmid pXL5105.

FIG. 9. Nucleic acid sequence (SEQ ID No. 41) (FIG. 9A) and amino acid sequence (SEQ ID No. 42) (FIG. 9B) of the HC antiAbeta_{—}13C13_F235A mAb for expression from expression plasmid pXL5111.

FIG. 10. Nucleic acid sequence (SEQ ID No. 43) (FIG. 10A) and amino acid sequence (SEQ ID No. 44) (FIG. 10B) of the HC antiAbeta_{—}13C13_V256A mAb for expression from expression plasmid pXL5132.

FIG. 11. Mass spectrometry data for AntiAbeta_{—}13C3 mAbs produced at different expression levels of glycosyltransferases. FIG. 11A, batch LP10081; FIG. 11B, batch LP10082; FIG. 11C, batch LP10084; FIG. 11D, batch LP10086.

FIG. 12. Mass spectrometry data for sialylated mAbs. FIG. 12A, spectrum of AntiAbeta_{—}13C3 (batch LP10088); FIG. 12B, spectrum of AntiAbeta_{—}13C3_V256A (batch LP10091); FIG. 12C, spectrum of AntiAbeta_{—}13C3_D257A (batch LP10094); FIG. 12D, spectrum of AntiAbeta_{—}13C3_F235A (batch LP10097), FIG. 12E, zoom in of FIG. 12A.

FIG. 13. Reactivity of AntiAbeta_{—}13C3 mAb variants (batches LP10088, LP10091, LP10094, LP10097) towards lectins MAA (FIG. 13A) and SNA (FIG. 13B) specific to α-2,3 and α-2,6 sialic acids in N-glycans, respectively.

FIG. 14. Reactivity towards lectins MAA (FIG. 14A) and SNA (FIG. 14B) of AntiAbeta_{—}13C3_D257A mAb produced in CHO in the presence of glycosyltransferases, SIAT1 and B4GT1.

FIG. 15. Reactivity of α-2,6 sialylated antiAbeta_{—}13C3_D257A towards SIGN-R1. ELISA towards SIGN-R1:Fc (coating: SIGN-R1:Fc [R&D Systems]; 2^{nd} antibody: anti mKappa-HRP).

FIG. 16. 16A, Reactivity of sialylated antiAbeta_{—}13C3 variants towards SIGN-R1. ELISA towards SIGN-R1:Fc (coating: SIGN-R1:Fc [R&D Systems]; 2^{nd} antibody: anti mKappa-HRP). AntiAbeta_{—}1303 and AntiAbeta_{—}13C3_D257A produced without or with B4GT1 and SIAT1 or SIAT6 glycosyltransferases (batches VA111018, VA111019, VA111026, VA111027 and VA111033); 16B: Reactivity of α-2,6 sialylated antiAbeta_{—}13C3 variants towards SIGN-R1. ELISA towards SIGN-R1:Fc (coating: SIGN-R1:Fc [R&D Systems]; 2^{nd} antibody: anti mKappa-HRP). AntiAbeta_{—}13C3, AntiAbeta_{—}13C3_D257A, AntiAbeta_F235A and AntiAntiAbeta_V256A mAb variants produced without or with B4GT1 and SIAT1 glycosyltransferases (batches VA111018 to VA 111029)

FIG. 17. Maps of expression plasmids pXL4973 coding for the light chain (LC) of humanized antiAbeta_{—}13C13_IgG4-D260X mAb where X=A, K, L, G or S (FIG. 17A), and pXL4979 coding for the heavy chain (HC) of humanized antiAbeta_{—}13C13_IgG4 mAb (FIG. 17B).

FIG. 18. Nucleic acid sequence (SEQ ID No: 3) and amino acid sequence (SEQ ID No. 4) of the LC of humanized antiAbeta_{—}13C13_D260X mAb where X=A, K, L, G or S.

FIG. 19. Nucleic acid sequence (SEQ ID No: 1) and amino acid sequence (SEQ ID No: 2) of the HC of humanized antiAbeta_{—}13C13_IgG4 mAb.

FIG. 20. Nucleic acid sequence (SEQ ID No: 47) and amino acid sequence (SEQ ID No: 48) of the HC of humanized antiAbeta_{—}13C13_IgG4-D260A mAb.

FIG. 21. Nucleic acid sequence (SEQ ID No: 53) and amino acid sequence (SEQ ID No: 54) of the HC of humanized antiAbeta_{—}13C13_IgG4-D260K mAb.

FIG. 22. Nucleic acid sequence (SEQ ID No: 51) and amino acid sequence (SEQ ID No: 52) of the HC of humanized antiAbeta_{—}13C13_IgG4-D260L mAb.

FIG. 23. Nucleic acid sequence (SEQ ID No: 49) and amino acid sequence (SEQ ID No: 50) of the HC of humanized antiAbeta_{—}13C13_IgG4-D260G mAb.

FIG. 24. Nucleic acid sequence (SEQ ID No: 55) and amino acid sequence (SEQ ID No: 56) of the HC of humanized antiAbeta_{—}13C13_IgG4-D260S mAb for expression.

FIG. 25. Mass spectrometry data for sialylated mAbs. FIG. 25A, spectrum of AntiAbeta_{—}13C3_IgG4 (batch VA1-11051); FIG. 25B, spectrum of AntiAbeta_{—}13C3_D2605 (batch VA1-11052); FIG. 25C, spectrum of AntiAbeta_{—}1303_D260G (batch VA1-11053); FIG. 25D, spectrum of AntiAbeta_{—}13C3_D260L (batch VA1-11054): FIG. 25E, spectrum of AntiAbeta_{—}13C3_D260K (batch VA1-11055); FIG. 25F, spectrum of AntiAbeta_{—}13C3_D260A (batch VA1-11056).

FIG. 26. Reactivity of AntiAbeta_{—}13C3_IgG4-D260X mAb variants (batches) towards lectins MAA (FIG. 26A) and SNA (FIG. 26B) specific to α-2,3 and α-2,6 sialic acids in N-glycans, respectively. Open lozenges: AntiAbeta_{—}13C3_IgG4 (batch VA1-11051); filled lozenges: AntiAbeta_{—}13C3_D260S (batch VA1-11052); open circles: AntiAbeta_{—}13C3_D260G (batch VA1-11053); open triangles: AntiAbeta_{—}13C3_D260L (batch VA1-11054); open squares: AntiAbeta_{—}13C3_D260K (batch VA1-11055); filled triangles: AntiAbeta_{—}13C3_D260A (batch VA1-11056); solid line: AntiAbeta_{—}13C3_D257A produced with B4GT1 and SIAT1 glycosyltransferases (batch LP 10104); dotted line: AntiAbeta_{—}13C3_D257A produced with B4GT1 and SIAT6 glycosyltransferases (batch VA-111033). dotted line+small filled circles: AntiAbeta_{—}13C3_D257A (batch LP 10106).

FIG. 27. Sequence alignment of IgG constant domains from human and murine isotype. The position of F243, of V264 and of D265 is highlighted with boxes. hIgG1 (SEQ ID NO: 57) corresponds to the constant domain of a human IgG1, as set forth in SwissProt entry No. IGHG1_HUMAN. hIgG2 (SEQ ID NO: 58) corresponds to the constant domain of a human IgG2. hIgG4 (SEQ ID NO: 59) corresponds to the constant domain of a human IgG4, as set forth in SwissProt entry No. IGHG4_HUMAN. hIgG4-PE (SEQ ID NO: 60) corresponds to the constant domain of a human IgG4 with a serine to proline substitution at position 228 and a leucine to glutamic acid substitution at position 235. mIgG1 (SEQ ID NO: 61) corresponds to the constant domain of a mouse IgG1 isolated from a hybridoma generated from BALBc mice. mIgG2a (SEQ ID NO: 62) corresponds to the constant domain of a mouse IgG2a. mIgG3 (SEQ ID NO: 63) corresponds to the constant domain of a mouse IgG3.

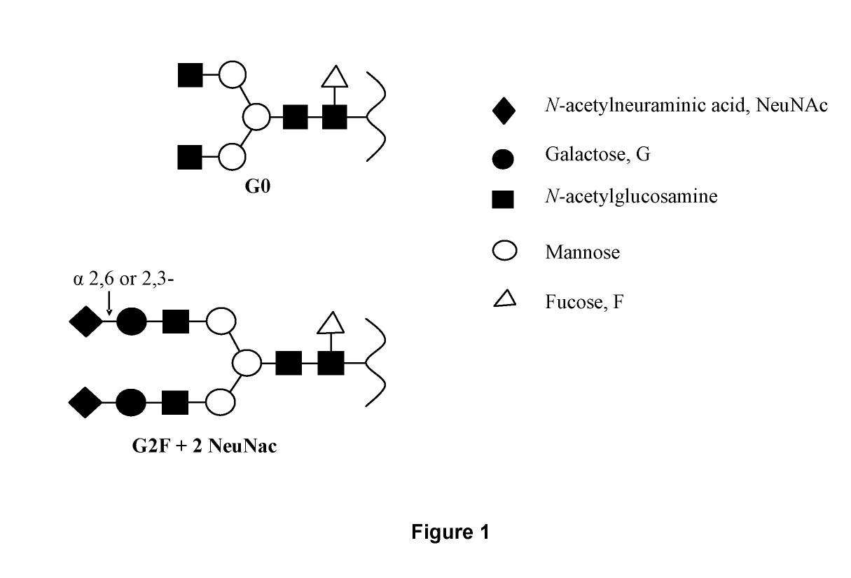

### EXAMPLES

In the following examples, the substitutions are referred to the positions on the amino acid sequence of the secreted polypeptide as provided in the figures and not by the EU numbering. Therefore position D265 in EU numbering corresponds to D257 on the HC antiAbeta_{—}13C13_D257A mAb or D260 on the HC of antiAbeta_{—}13C13_IgG4-D260A mAb, antiAbeta_{—}13C13_IgG4-D260K, antiAbeta_{—}13C13_IgG4-D260L mAb, antiAbeta_{—}13C13_IgG4-D260G mAb, antiAbeta_{—}13C13_IgG4-D260S mAb. Similarly F243A in EU numbering corresponds to F235A on the HC antiAbeta_{—}13C13_F235A mAb, and V264A in EU numbering corresponds to V256 on the HC antiAbeta_{—}13C13_V256A mAb.

### Example 1

### Low mAb Productivity when Glycosyltransferases are Overexpressed

In this example, the transient production of a monoclonal antibody (mAb) in the presence of glycosyltransferases was shown to decrease significantly while the concentration of plasmids encoding these glycosyltransferases increased.

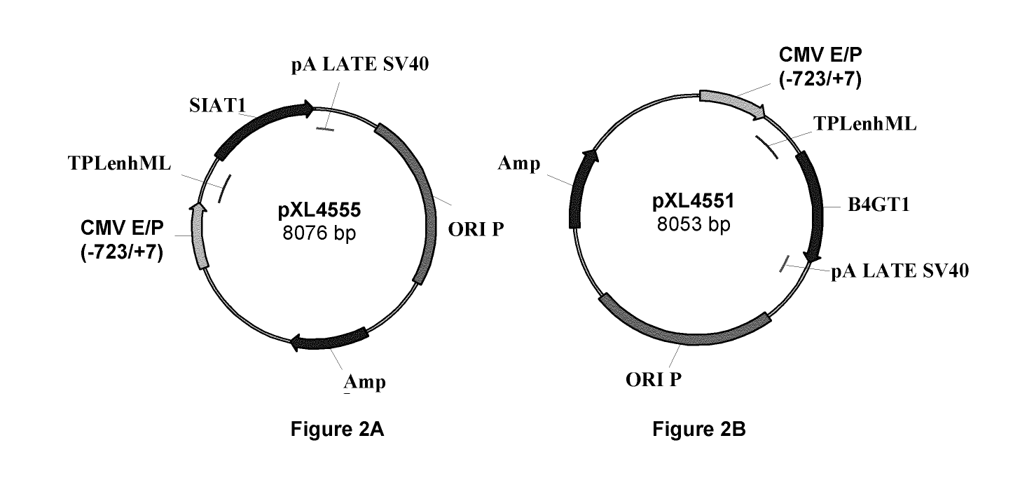

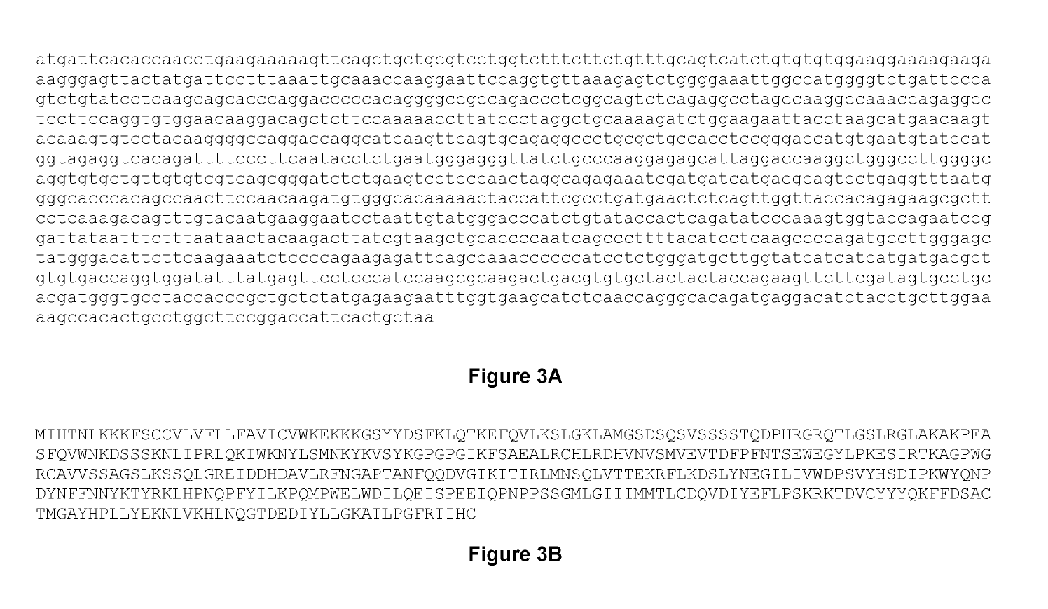

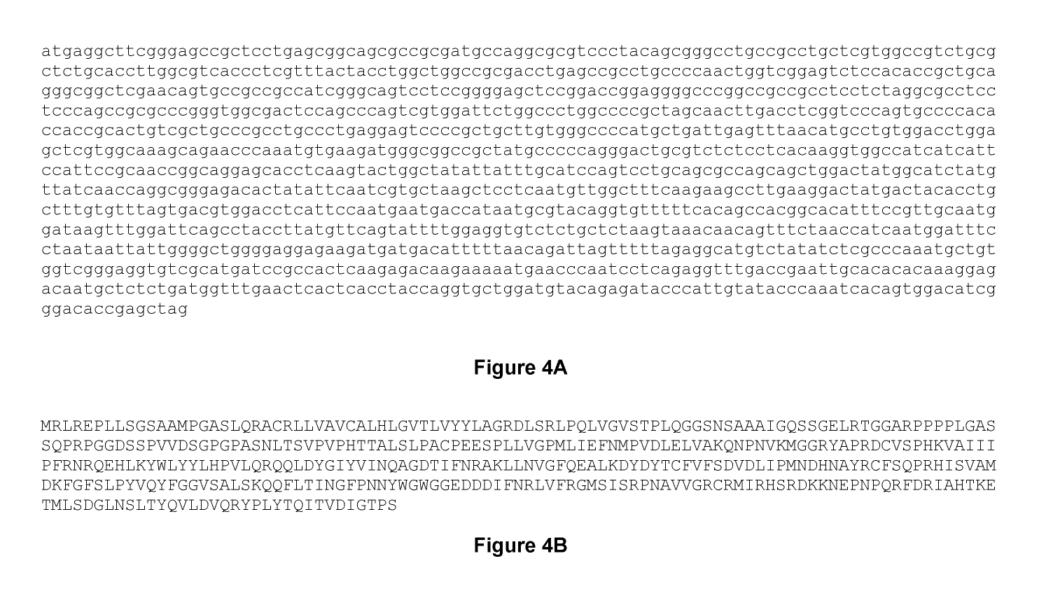

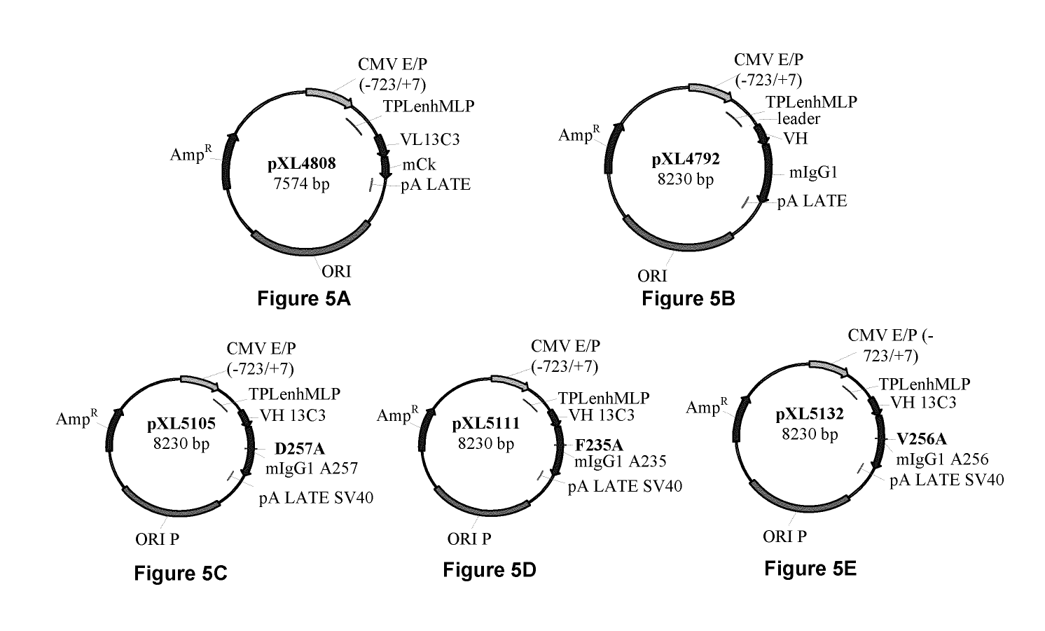

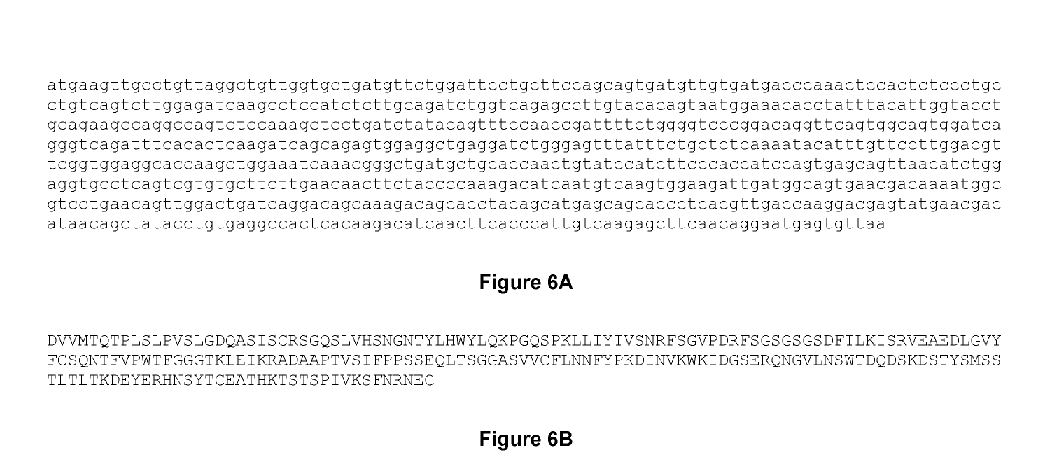

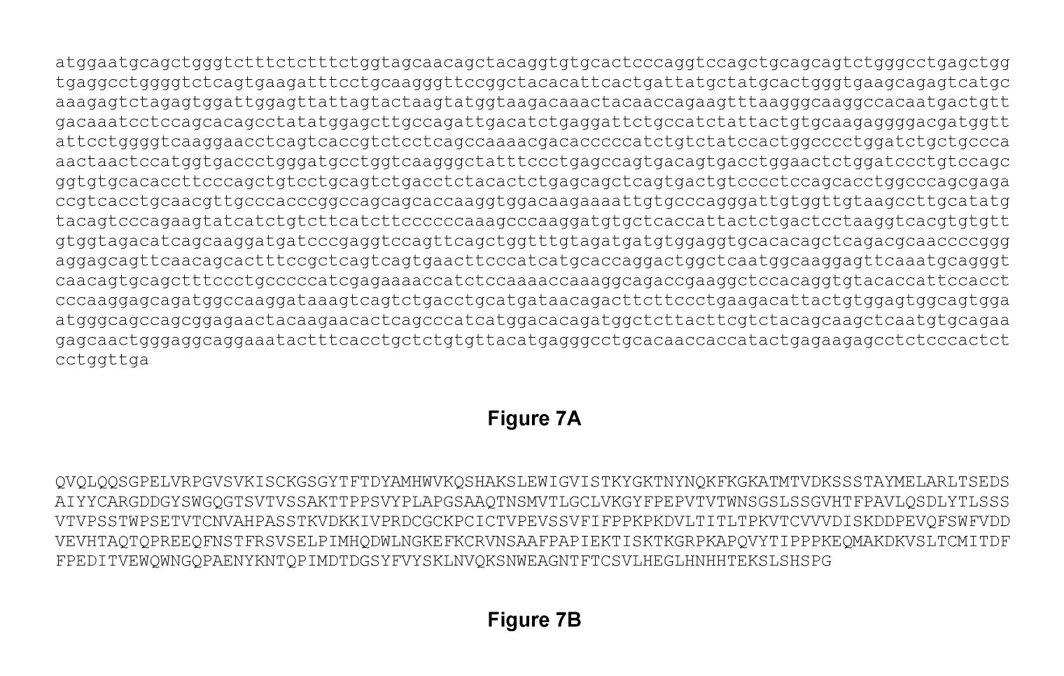

The cDNAs encoding human α-2,6 sialyltransferase (SIAT1) (SEQ ID No. 33) or human β-1,4 galactosyltransferase (B4GT1) (SEQ ID No. 35) were retrieved from a clone collection (Invitrogen) and inserted into the mammalian expression vector pXL4214 from which expression is driven from the CMV promoter to generate plasmids pXL4555 and pXL4551. Maps of plasmid are presented on FIG. 2, the nucleic acid and corresponding amino acid sequences of SIAT1 and B4GT1 are on FIGS. 3 and 4 respectively. The same expression vector was also used to clone the cDNA encoding the light chain (LC) and heavy chain (HC) of the murine AntiAbeta_{—}13C3 mAb. Plasmid pXL4808 encoded LC of antiAbeta_{—}13C13 mAb, FIG. 5A; Plasmid pXL4792 encoded HC of antiAbeta_{—}13C13 mAb, FIG. 5B. The LC was the murine Ckappa and the HC the murine IgG1 isotype. The nucleic acid and corresponding amino acid sequences of the LC and HC mAb variants were described on FIGS. 6 and 7. (SEQ ID No. 37 to 40)

Transient expression of the AntiAbeta_{—}13C3 mAb was performed in suspension-cultivated 293-F cells (derived from human embryonic kidney HEK 293 cells and purchased at Invitrogen) by co-transfection of four plasmids pXL4792, pXL4808, pXL4551 and pXL4555 complexed with 293Fectin™ (Invitrogen) at different ratios. A plasmid encoding EBNA was also included as reported by Durocher et al. (Nucl. Acids Res., 30: e9, 2002). Cell culture and transfections were performed according to the recommendations from the supplier (Invitrogen) in shake flasks at 100 mL scale. Eight days post transfection, viable cells were counted (Vi-CELL XR Cell Viability Analyzer (Beckman Coulter)) and mAb concentrations were determined by analytical HPLC (Poros G/20) coupled to UV detection at 280 nm. As shown in Table 2, mAb production corresponded to cell harvested when viable cells significantly decreased.

When the concentration of plasmids encoding SIAT1 and B4GT1 was increased by a factor of 40, percentage of viable cells decreased and productivity dropped by a factor of 5 (see Table 2).

TABLE 2
mAb productivity in the presence of glycosyltransferases

| | Ratio of plasmid encoding | | | | Viable | |
|---|---|---|---|---|---|---|
| | LC and | | | | cells | Production |
| Batch | HC | SIAT1 | B4GT1 | Ballast | % | mg/L |
| LP10081 | 6 | 0 | 0 | 4 | 63 | 54 |
| LP10082 | 6 | 0.05 | 0.05 | 3.9 | 60 | 57 |
| LP10083 | 6 | 0.15 | 0.15 | 3.7 | 59 | 52 |
| LP10084 | 6 | 0.5 | 0.5 | 3 | 52 | 30 |
| LP10085 | 6 | 1 | 1 | 2 | 47 | 17 |
| LP10086 | 6 | 2 | 2 | 0 | 43 | 11 |

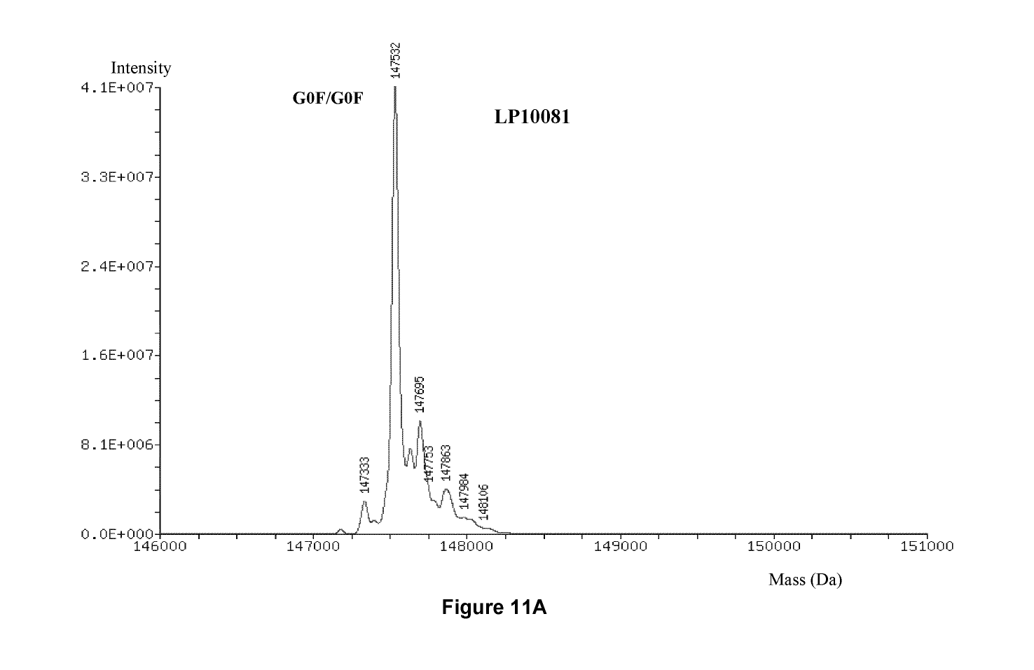

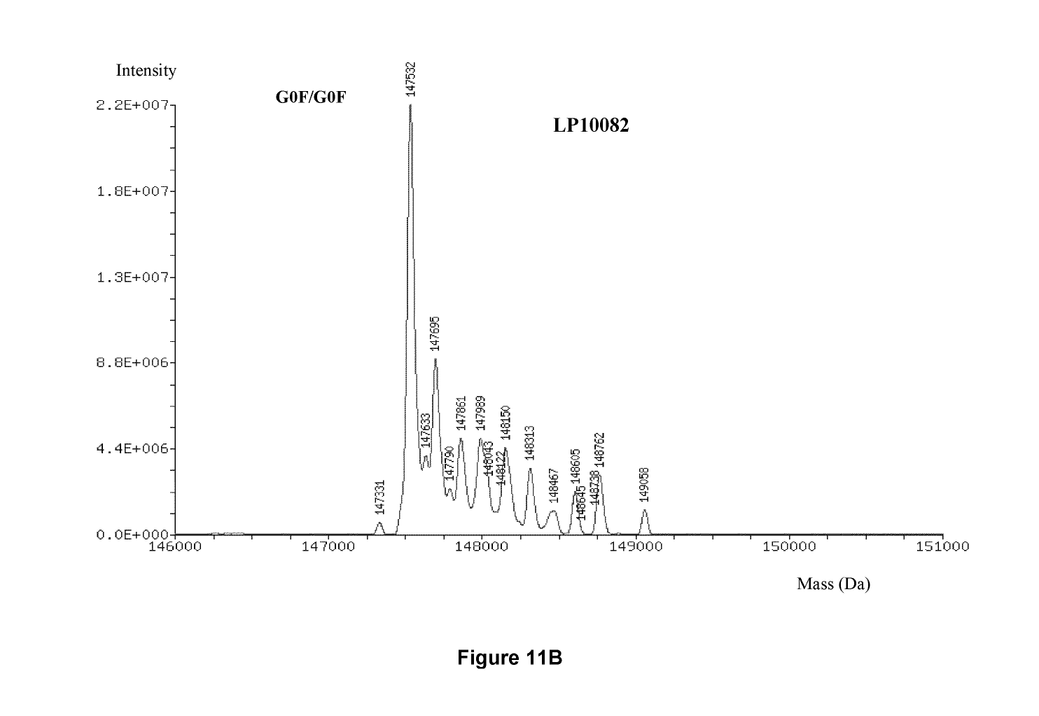

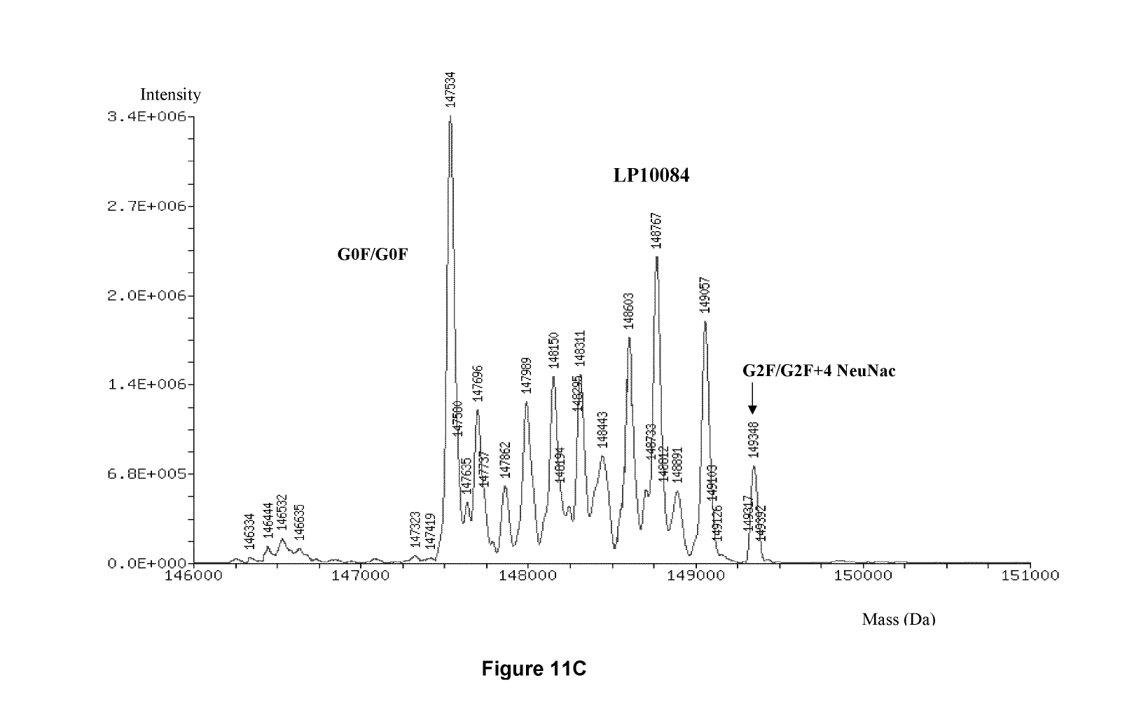

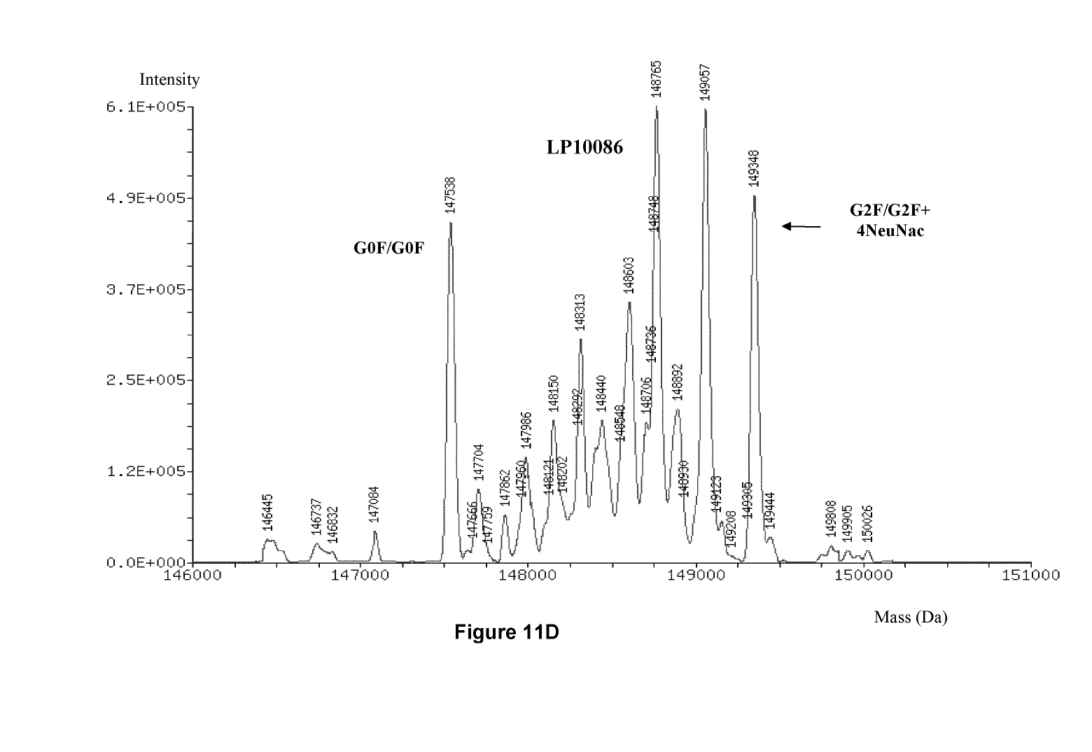

The six mAbs batches were purified by affinity chromatography on Protein A (MabSelect, GE, Healthcare) and eluted from the column with 100 mM acetic acid pH 2.8, 20 mM NaCl buffer. They were formulated in PBS and analyzed by mass spectrometry on nanoLC coupled to LTQ-Orbitrap MS. The expected mass of antiAbeta_{—}13C3 mAb and the presence of N-glycans are shown on FIG. 11. When the expression levels of the glycosyltransferases increased, the sialylated content of the N-glycan was higher and more complex.

### Example 2

### Production of mAb Variants with α-2,6-Sialylated N-Glycan in Fc

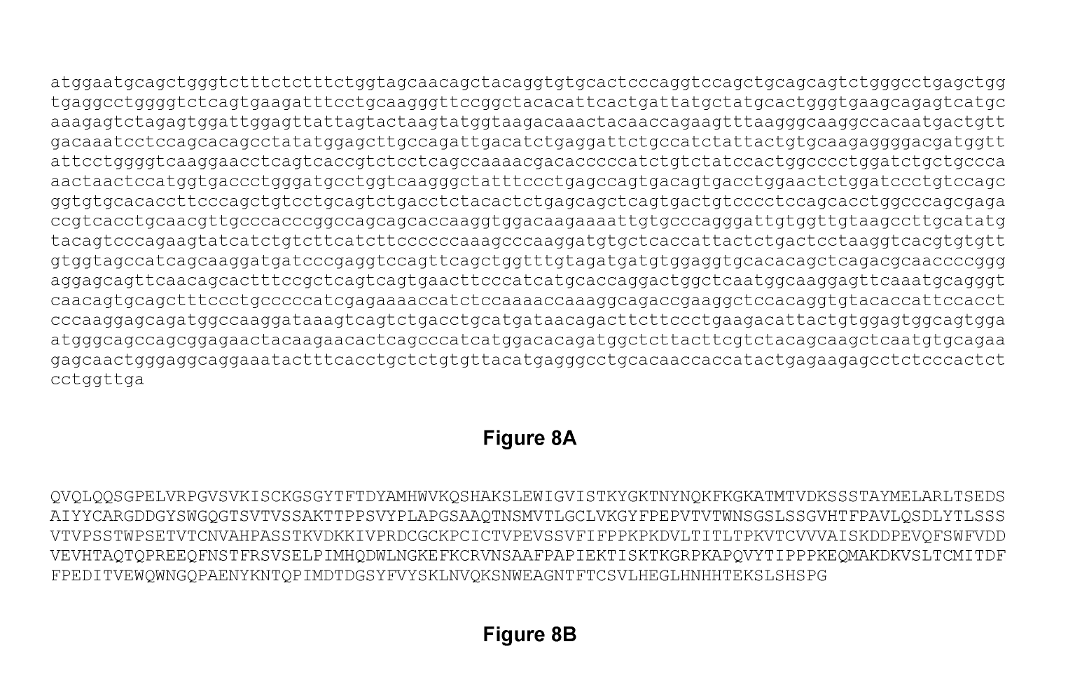

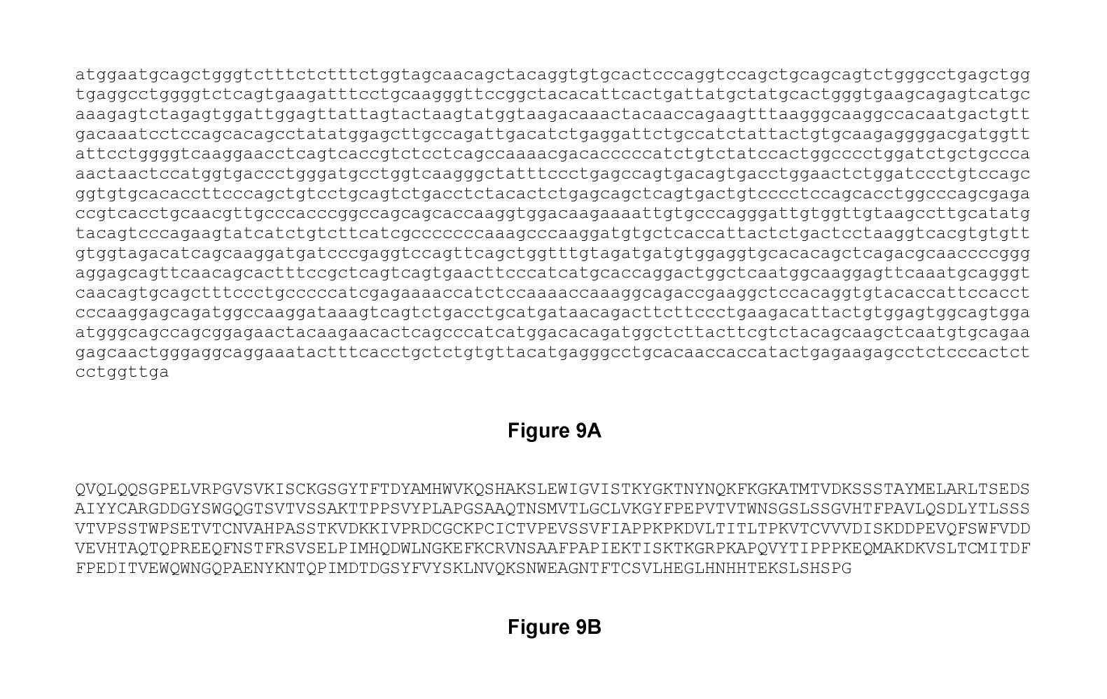

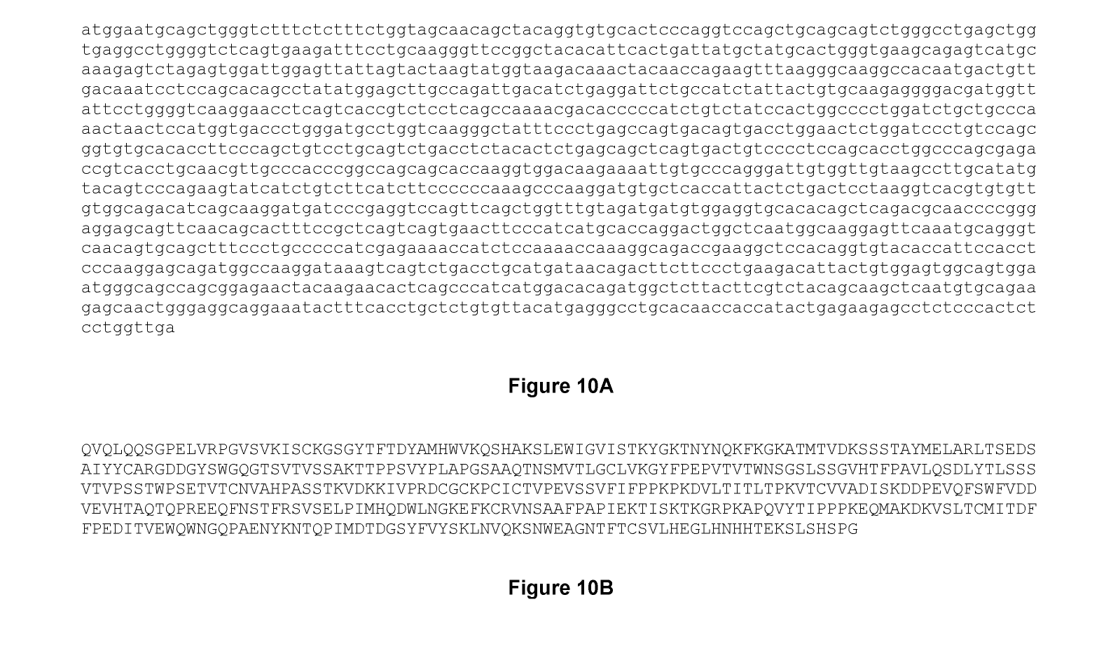

In this example, the production of mAb variants with α-2,6-sialylated N-glycan in Fc is described by transient expression in mammalian cells HEK 293 or CHO at small scale. The same expression vector was used to clone the cDNA encoding LC and HC of AntiAbeta_{—}13C3 mAb variants. The following plasmids were generated and were shown on FIG. 5. Plasmid pXL4808 encoded LC of antiAbeta_{—}13C13 mAb, FIG. 5A; Plasmid pXL4792 encoded HC of antiAbeta_{—}13C13 mAb, FIG. 5B; Plasmid pXL5105 encoded the modified HC of AntiAbeta_{—}13C3_D257A, FIG. 5C; Plasmid pXL5111 encoded the modified HC of AntiAbeta_{—}13C3_F235A mAb, FIG. 5D and plasmid pXL5132 encoded the modified HC of AntiAbeta_{—}13C3_V256A mAb, FIG. 5E. The nucleic acid and corresponding amino acid sequences of the LC and HC mAb variants were described on FIGS. 6, 7, 8, 9 and 10. The nucleotide sequences of the HC AntiAbeta_{—}13C3_F235A, AntiAbeta_{—}13C3_V256A, and AntiAbeta_{—}13C3_D257A mAb variants correspond to the sequences SEQ ID NOS: 41, 43, and 45, respectively. The amino acid sequences of the HC AntiAbeta_{—}13C3_F235A, AntiAbeta_{—}13C3_V256A, and AntiAbeta_{—}13C3_D257A mAb variants correspond to the sequences SEQ ID NOS: 42, 44, and 46, respectively. Positions 235, 256, and 257 of the murine IgG1 Fc domain correspond respectively to positions 243, 264, and 265 in the human IgG1 Fc domain using the EU numbering.

Each monoclonal antibody variant was produced in suspension-cultivated 293-F cells by transient co-expression of four plasmids encoding the HC, LC, SIAT1 and B4GT1 complexed with 293Fectin™ (Invitrogen). The plasmid ratio was optimized to ensure optimal productivity and sialic acid content. The optimal plasmid ratio was 6/0.5/0.5 for [HC and LC plasmids]/[SIAT1 plasmid]/[B4GT1 plasmid]. The secreted mAbs were harvested eight days post transfection and centrifuged. The mAbs were purified by affinity chromatography on Protein A (MabSelect, GE, Healthcare) and eluted from the column with 100 mM acetic acid pH 2.8, 20 mM NaCl buffer. They were formulated in PBS, 0.22 μm-filtered and stored at +5° C. Purified mAb concentrations were determined by measurement of absorbance at 280 nm.

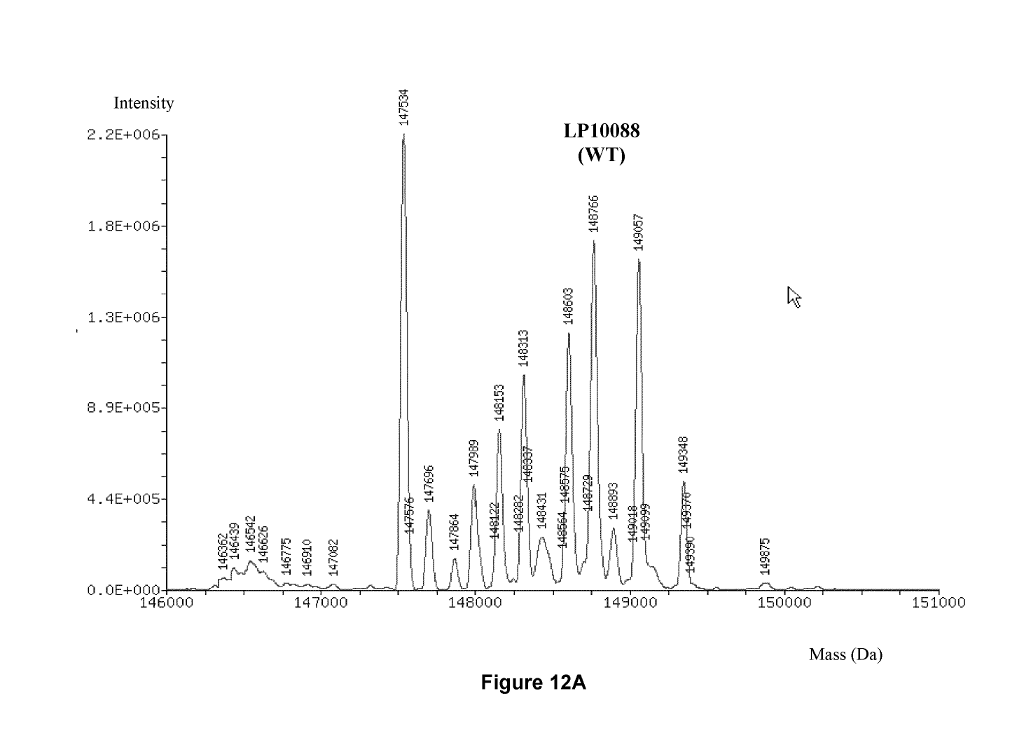

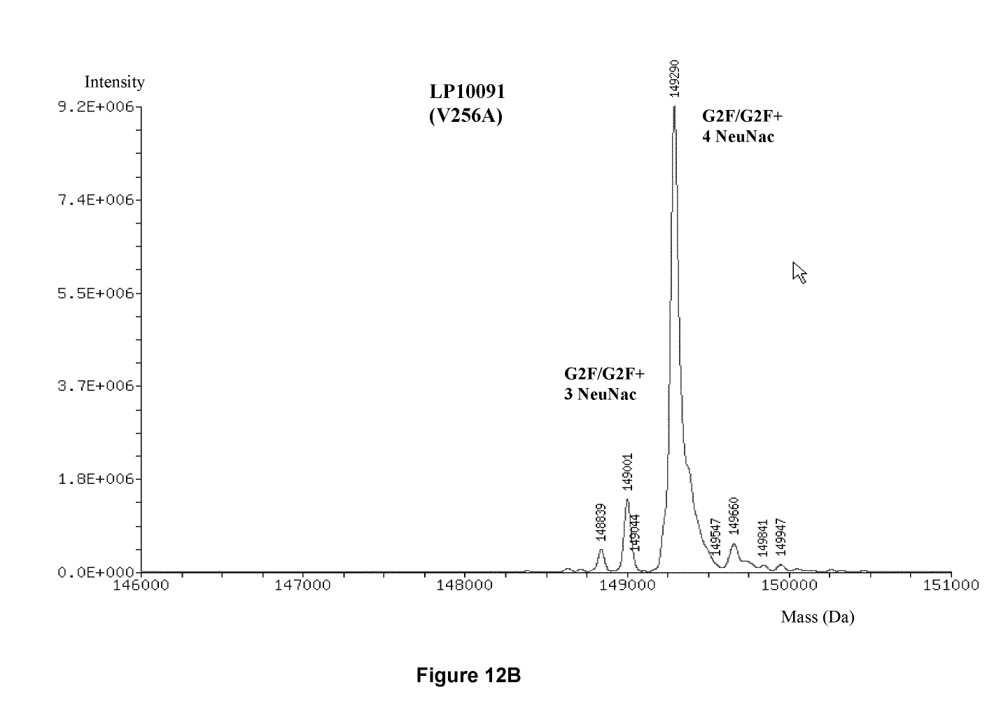

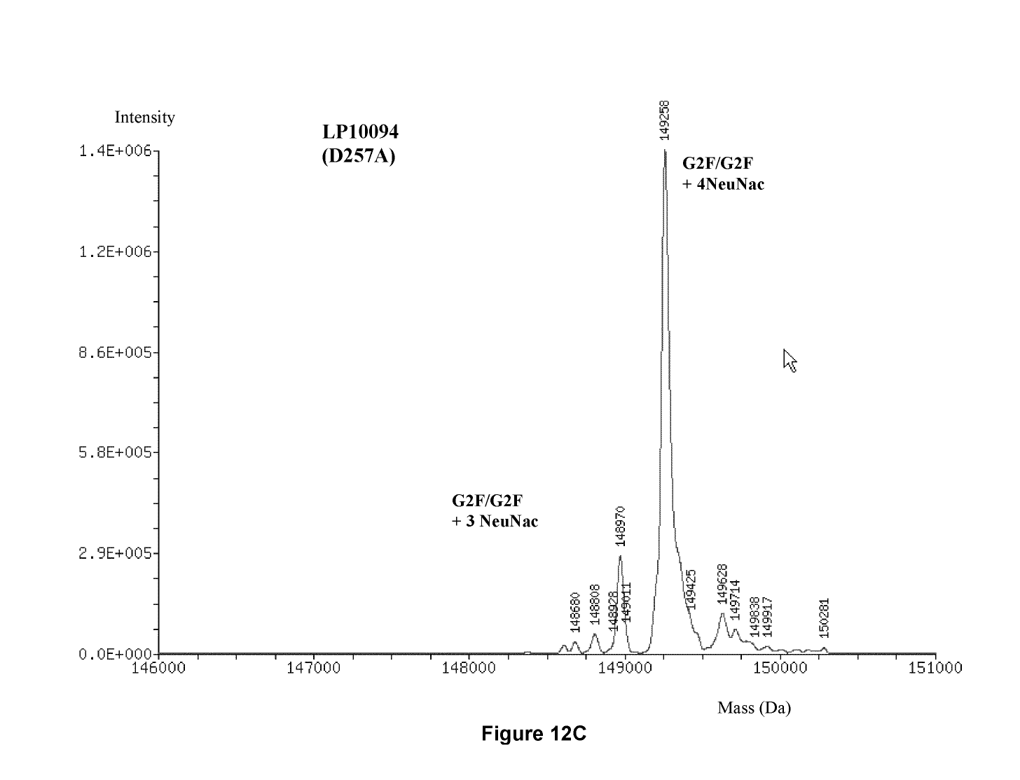

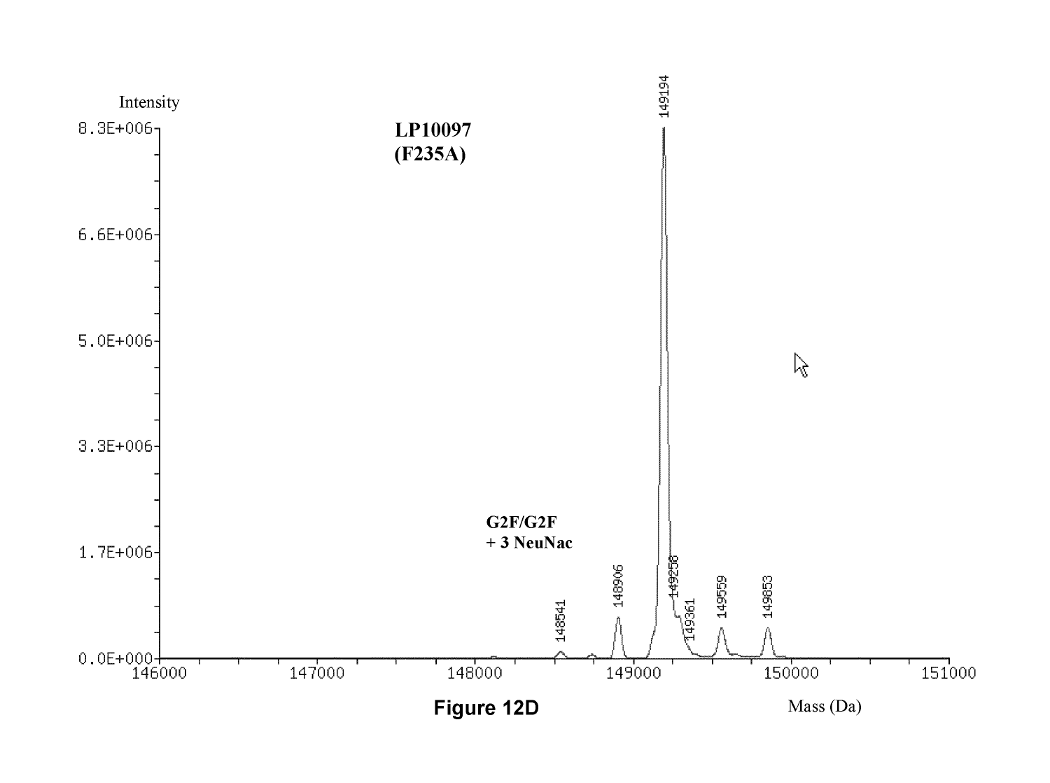

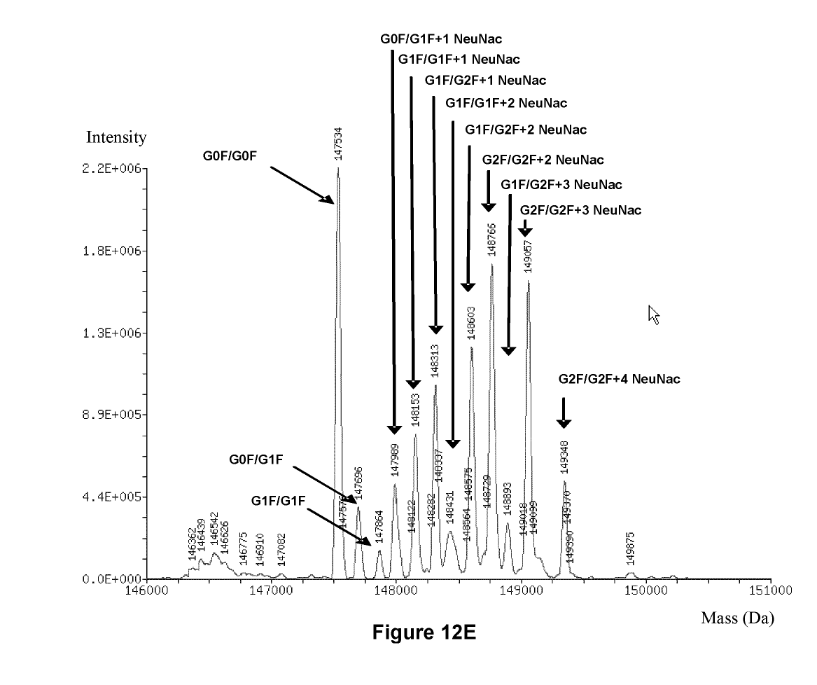

A total of 1.5 to 1.8 mg of mAb was purified from 150 mL culture. Each batch was analyzed by SDS-PAGE (Nupage Bistris/MOPS-SDS 4-12%, Invitrogen) under reducing and non-reducing conditions to determine a purity of more than 99% and the expected molecular weight of each subunit and of the monomer. Each batch was also analyzed by gel filtration (Tricorn 10/300 GL Superdex 200) to determine the homogeneity of the monomer at 99% and the low content of high molecular weight species of less than 1.2%. Mass spectrometry analysis was carried out on nanoLC coupled to LTQ-Orbitrap MS. It revealed the expected mass of the different mAbs and the N-glycans essentially sialylated with each variant containing a point mutation in the Fc domain for batches LP10091, 10094, and LP10097 (see FIG. 12 and Table 3).

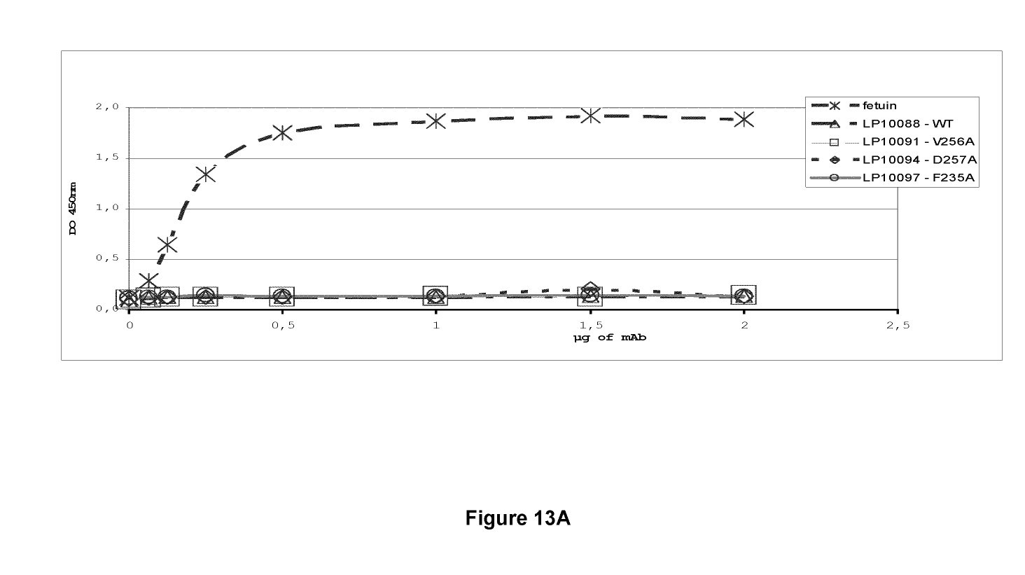

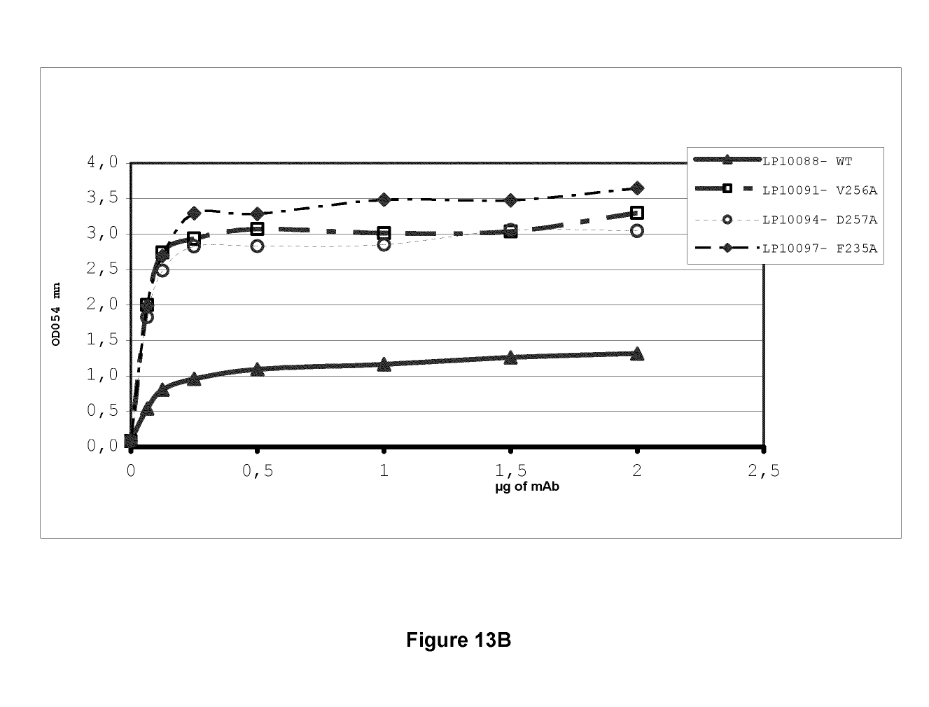

Two enzyme-linked lectin assays (ELLA) were developed to detect either terminal α-2,3 sialic acid in N-glycan with lectin Maackia amurensis (MAA) or terminal α-2,6 sialic acid in N-glycan with lectin Sambucus nigra (SNA). As shown on FIG. 13, no reactivity was found to MAA whereas specificity was observed with SNA and reactivity was higher when the sialylated content of the N-glycan was higher (see batches LP10091, LP10094 and LP10097).

TABLE 3
Characteristics of mAb variants with α-2,6-sialylated N-glycan in Fc

| | | | | Mass Spectrometry | | Reactivity | |
|---|---|---|---|---|---|---|---|
| | | Plasmids | mAb | | Theoretical mass | towards | |
| | | LC and | purified | Mass | of mAb with N- | MAA | SNA |
| Mutation | Batch | HC | (mg) | (Da) | glycan as | α-2,3 | α-2,6 |
| Wild-type | LP10088 | pXL4808 | 1.6 | 147534 | G0F/G0F | no | Intermediate |
| | | pXL4792 | | (major) | G2F/G2F + 4 | | |
| | | | | 149347 | NeuNAc + | | |
| | | | | (minor) | at least 10 | | |
| | | | | | additional | | |
| | | | | | species with 0 to | | |
| | | | | | 3 NeuNAc | | |
| V256A | LP10091 | pXL4808 | 1.7 | 149290 | G2F/G2F + 4 | no | high |
| | | pXL5132 | | (major) | NeuNAc | | |
| | | | | 149001 | G2F/G2F + 3 | | |
| | | | | (minor) | NeuNAc | | |
| D257A | LP10094 | pXL4808 | 1.8 | 149258 | G2F/G2F + 4 | no | high |
| | | pXL5105 | | (major) | NeuNAc | | |
| | | | | 148970 | G2F/G2F + 3 | | |
| | | | | (minor) | NeuNAc | | |
| F235A | LP10097 | pXL4808 | 1.5 | 149194 | G2F/G2F + 4 | no | high |
| | | pXL5111 | | (major) | NeuNAc | | |
| | | | | 148906 | G2F/G2F + 3 | | |
| | | | | (minor) | NeuNAc | | |

Taken together, these results indicated that, when mAb variants engineered with one of the three point mutations in the Fc (V256A, D257A, F235A) were produced by transient expression in HEK293 cells in the presence of plasmids encoding B4GT1 and SIAT1, N-glycans consisted essentially of α-2,6-sialylated forms. More specifically, the presence of V256A, D257A or F235A leads to the obtention of antibodies species that exhibit a very homogeneous sialylation profile (see FIGS. 12B, C and D), said species being fully characterized and defined (see Table 3). The major peak, which is really dominant compared to the other peaks, corresponds to a species that is fully silylated (four sialic acid residues). In contrast to this, overexpression of B4GT1 and SIAT1 with wild-type mAb resulted in the production of a mixture of at least 12 different species containing non-sialylated or incompletely sialylated N-glycans (FIG. 12A).

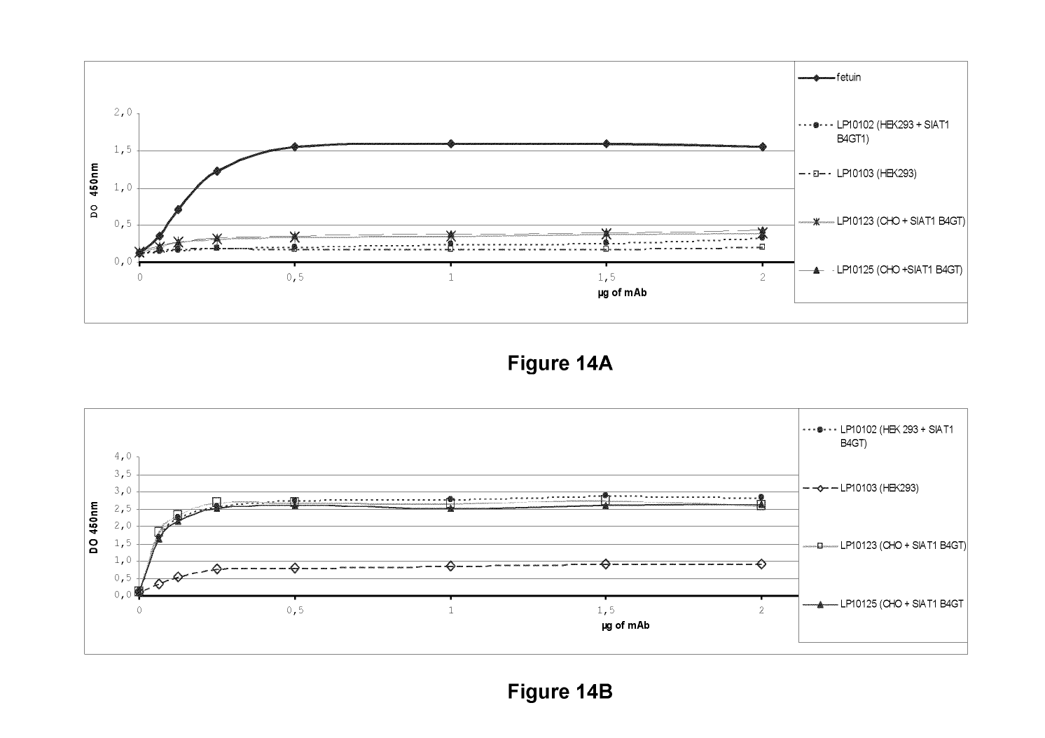

An antiAbeta_{—}13C3_D257A mAb variant was also produced in suspension-cultivated CHO cells by transient co-expression of the four plasmids encoding the HC pXL5105, LC pXL4808, SIAT1 pXL4555 and B4GT1 pXL4551 with the optimal plasmid ratio used in HEK293. Similar content of α-2,6 sialic acid was detected by ELLA assays with the batches produced in CHO and HEK 293, see FIG. 14.

### Example 3

### Large Scale Production of mAb Variant with α-2,6-Sialylated N-Glycan in Fc

In this example, the production of antiAbeta_{—}13C3_D257A mAb with α-2,6-sialylated N-glycan in Fc is described by transient co-expression with SIAT1 and B4GT1 in mammalian cells at large scale. Characterization and binding specificities of this mAb were compared to the same antiAbeta_{—}13C3_D257A mAb produced without co-expression of SIAT1 and B4GT1.

AntiAbeta_{—}13C3_D257A mAb variant was produced in suspension-cultivated 293-F cells in 10-L Wave Bioreactor by transient co-expression of the four plasmids encoding the HC (pXL5105), LC (pXL4808), SIAT1 (pXL4555) and B4GT1 (pXL4551) complexed with 293Fectin™, using the optimal plasmid ratio used in Example 1. The batch was harvested 8 days post transfection and named LP10104. Another batch named LP10116 was also produced in suspension-cultivated 293-F cells in 10-L Wave Bioreactor by transient co-expression of the plasmids encoding the HC (pXL5105) and the LC (pXL4808). Both batches were purified and characterized as described in Example 1. The characterization of the two batches LP10104 and LP10116 is summarized in Table 5.

Quantitative sialic acid identification, carbohydrate composition analysis and quantitative oligosaccharide mapping of N-glycans in the mAbs were also performed essentially as described previously (Saddic et al., Methods Mol. Biol., 194: 23-36, 2002; Anumula et al., Glycobiology, 8: 685-694, 1998). First, sialic acid residues were released after mild hydrolysis of mAb and fluorescently labeled with ortho-phenylenediamine and separated by reversed-phase HPLC. Individual peaks were detected by fluorescence detection (excitation, 230 nm; emission, 425 nm), identified and quantified by comparison with N-acetylneuraminic (NeuNAc) and N-glycolylneuraminic (NeuNGc) acid standards. Second, the carbohydrate composition was determined after acid hydrolysis of mAb samples to release the individual monosaccharides. After hydrolysis, the monosaccharides (neutral and amino sugars) were derivatized with anthranilic acid and then separated by reversed-phase HPLC and detected by fluorescence detection (excitation, 360 nm; emission, 425 nm). Individual peaks were identified and quantified by comparison with monosaccharide standards. Third, oligosaccharides were enzymatically released with PNGase F and fluorescently labeled with anthranilic acid before separation according to their number of sialic acid residues by normal phase-anion exchange HPLC on an Asahipak-NH2P (Phenomenex) column. Labeled glycans were detected and quantified by fluorescence detection (excitation, 360 nm; emission, 425 nm). Analytical data are reported on Table 4.

TABLE 4
Analytical content of N-glycans on batches LP10104 and LP10116

| | Sialic acids (SA) | | | Monosaccharides | | | | | | |
|---|---|---|---|---|---|---|---|---|---|---|
| | | | Number/ | number of sugar/ | | | Glycan | | | |
| | mol/mol protein //% | | glycan | 3 mannoses | | | Mapping | | | |
| Batch | NeuNAc | NeuGc | SA | GlcN | Gal | Fuc | 0SA | 1SA | 2SA | 3SA |
| LP10104 | 1.5//100% | Not detected | 1.5 | 4.37 | 2.04 | 1.04 | 17 | 18.5 | 64 | 0.5 |
| LP10116 | 0.13//100% | Not detected | 0.13 | 4.87 | 1.02 | 1.12 | 87 | 11 | 2 | 0 |

TABLE 5
Characteristics of LP10104 large batch of AntiAbeta_13C3_D257A mAb
variant with α-2,6 sialylated N-glycan in Fc.

| Production, Purification | | |
|---|---|---|
| Characterization Process | LP10104 | LP10116 |
| Transient expression in | HEK 293 10L-batch | HEK 293 10L-batch |
| Cotransfection with | Plasmids encoding SIAT1 and B4GT | none |
| | glycosyltransferases | |
| Purification steps | Protein A affinity | Protein A affinity |
| | | CHT type I |
| Formulation | PBS | PBS |
| Concentration (mg/mL) | 4.01 | 4.65 |
| Purified Quantity (mg) | 169 | 669 |
| Mass by Mass | 149258 | 147445 |
| Spectrometry (Da) | | |
| Glycan analysis by Mass | G2F/G2F+ 4 NeuNAc | G0F/G0F |
| Spectrometry | | |
| Affinity to lectins (SNA | specific to α-2,6 sialic acid | No affinity detected |
| and MAA) | | |
| Quantitative sialic acid | More than 1.5 sialyl group per glycan | Less than 5% of the |
| identification by analytical | Around 90% of the mAbs having at | mAbs having at least |
| HPLC | least one disialylated N-glycan | one disialylated N- |
| | Predominantly bi-antennary-α2,6 | glycan |
| | disialyl N-glycan | |
| | No N-glycolylneuraminic acid detected | |
| Purity by SDS-PAGE | 99% | 99% |
| % aggregates | 0.2% | Not detected |
| Endotoxin level (LAL) | 0.07 | 0.04 |
| EU/mg | | |
| Sterility test | Conform | Conform |

The overall data presented in this example show that hundreds of milligrams of AntiAbeta mAb with very high content of α-2,6 sialylated N-glycans Fc can be produced with the quality required for therapeutic usage. This mAb has been named α-2,6 sialylated antiAbeta_{—}13C3_D257A in the following examples.

### Example 4

### Affinity of α-2,6 Sialylated antiAbeta_{—}13C3_D257A Towards its Ligand

In this example, affinity of antiAbeta_{—}13C3_D257A to Aβ protofibrils was assayed since the original antiAbeta_{—}13C3 mAb binds specifically to this ligand.

Protofibrils are soluble rod-like structures derived from the amyloid beta peptide Aβ1-42 peptide by self aggregation. They were obtained by dissolving the synthetic human Aβ1-42 peptide in 10 mM NaOH and incubation in NaCl/Phosphate buffer for 16 hours at 37° C. as previously published (Johansson et al., FEBS Journal, 273: 2618-30, 2006). Protofibrils with molecular weight higher than 200 kDa were separated by Size Exclusion Chromatography from low molecular weight forms with molecular weight of around 11 kDa. Affinity was assayed by ELISA, protofibrils were coated onto 96-well plates, a concentration range of antibodies was applied and detection was performed with anti-Fc monoclonal antibodies coupled to peroxidase.

Affinity of Aβ protofibrils to α-2,6 sialylated antiAbeta_{—}13C3_D257A was measured with an EC₅₀ of 0.0415 mg/L, similar to the EC₅₀ obtained with the original antiAbeta_{—}13C3 and to the low sialylated antiAbeta_{—}13C3_D257A, as described on Table 6.

Therefore, the modification due to the α-2,6 sialylated N-glycans Fc did not interfere with the mAb/ligand affinity.

TABLE 6
Affinity of α-2,6 sialylated antiAbeta_13C3_D257A to Aβ protofibrils

| | | Sialic acid | EC₅₀ to PF |
|---|---|---|---|
| mAb | Batch | content | (mg/L) |
| antiAbeta_13C3 | LP09009 | low | 3.84E−02 |
| antiAbeta_13C3_D257A | LP10104 | Very high | 4.15E−02 |
| antiAbeta_13C3_D257A | LP10116 | low | 3.70E−02 |

### Example 5

### Affinity of α-2,6 Sialylated antiAbeta_{—}13C3_D257A Towards the Fcγ Receptors

The α-2,6 sialylated antiAbeta_{—}13C3_D257A mAb described in Example 3 has been significantly modified in the Fc domain by the presence of extensively sialylated N-glycans. This modification could interfere with the Fc binding to the Fcγ receptors and C1q component that are described to bind in this domain (Shields et al. J. Biol. Chem., 276: 6591-6604, 2001; Mershon et al., pages 373-382, “Therapeutic monoclonal antibodies: from bench to clinic”, Ed.: Zhiqiang An, 2009, John Wiley & Sons, Inc., Hoboken, N.J., USA). Therefore affinities of α-2,6 sialylated antiAbeta_{—}13C3_D257A were determined toward murine proteins FcγRs and C1q in comparison to a murine IgG2a monoclonal antibody (LP09078) with potent Fc-mediated effector functions.

Affinities of α-2,6 sialylated antiAbeta_{—}13C3_D257A towards recombinant murine FcγRs (obtained from R&D Systems) were determined with Surface Plasmon Resonance technology (SPR) using a Biacore 3000 instrument. Affinity data were analyzed with BiaEvaluation software. Affinity parameters were determined either with steady state analysis for low affinity with fast dissociation, or with global fit with appropriate model for high affinity with slow dissociation.

Affinity of α-2,6 sialylated antiAbeta_{—}13C3_D257A towards recombinant C1q was measured by ELISA. Recombinant C1q from Calbiochem (reference 204876), was coated onto 96-well plates, a concentration range of antibodies was applied and detection was performed with anti-Fc monoclonal antibodies coupled to peroxidase. Results indicated in Table 7 showed that the affinities of antiAbeta_{—}13C3_D257A towards FcγR and C1q were very low in the absence and in the presence of α-2,6 sialylated N-glycans Fc.

The modification due to the α-2,6 sialylated N-glycans Fc did not interfere with the mAb affinities to the Fcγ Receptors nor the C1q component. Therefore the ability for engaging the immune effector cells or the complement cascade would be very low with this α-2,6 sialylated antiAbeta_{—}13C3_D257A.

TABLE 7
Affinity of α-2,6 sialylated antiAbeta_13C3_D257A
to Fcγ receptors and C1q component.

| | | | | | | | C1q |
|---|---|---|---|---|---|---|---|
| Characteristics of mAb | | | | | | | component |
| | | Sialic | | | | | (EC₅₀, |
| | | acid | FcγReceptor (K_{D}) | | | | mg/L) |
| Name | Batch | content | FcγRI | FcγRIIb | FcγRIII | FcγRIV | C1q |
| antiAbeta_13C3 | LP09009 | Low | No | 354 nM | 471 nM | No | No binding |
| | | | binding | | | binding | |
| antiAbeta_13C3_D257A | LP10104 | Very | No | >4 μM | >2.3 μM | No | No binding |
| | | high | binding | | | binding | |
| antiAbeta_13C3_D257A | LP10116 | low | No | >5.3 μM | >1.9 μM | No | No binding |
| | | | binding | | | binding | |
| antiAbeta_13C3_mIgG2a | LP09078 | low | 15.2 μM | 704 μM | 349 μM | 14.3 nM | 26.9 |

### Example 6

### Affinity of α-2,6 Sialylated antiAbeta_{—}13C3_D257A Towards SIGN-R1

It had been hypothesized that α-2,6 sialylated Fc engaged SIGN-R1, a lectin that induced a cellular program resulting in the secretion of anti-inflammatory, soluble mediators that target effector macrophages (Anthony et al., Proc Natl Acad Sci U.S.A., 105: 19571-19578, 2008). Therefore, in this example, the affinity of α-2,6 sialylated antiAbeta_{—}13C3_D257A to SIGN-R1 was assayed.

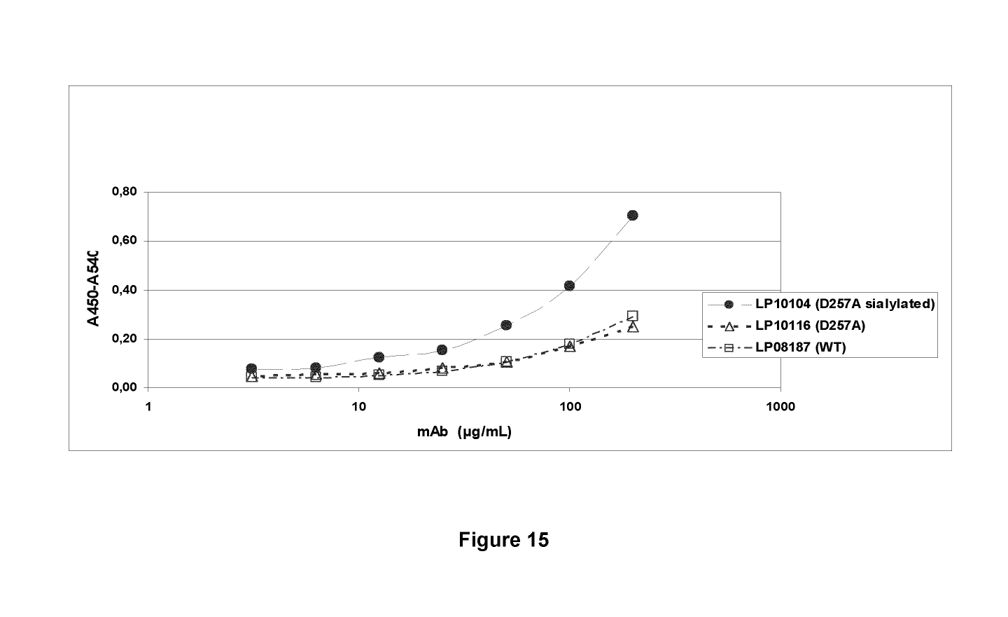

Affinity was assayed by ELISA: SIGN-R1::Fc obtained from R&D Systems was coated onto 96-well plates, a concentration range of antibodies was applied and detection was performed with anti-murine Ckappa monoclonal antibodies coupled to peroxidase. Results presented on FIG. 15 indicate that α-2,6 sialylated antiAbeta_{—}13C3_D257A (batch LP10104) had more reactivity to SIGN-R1 than antiAbeta_{—}13C3_D257A (batch LP10116).

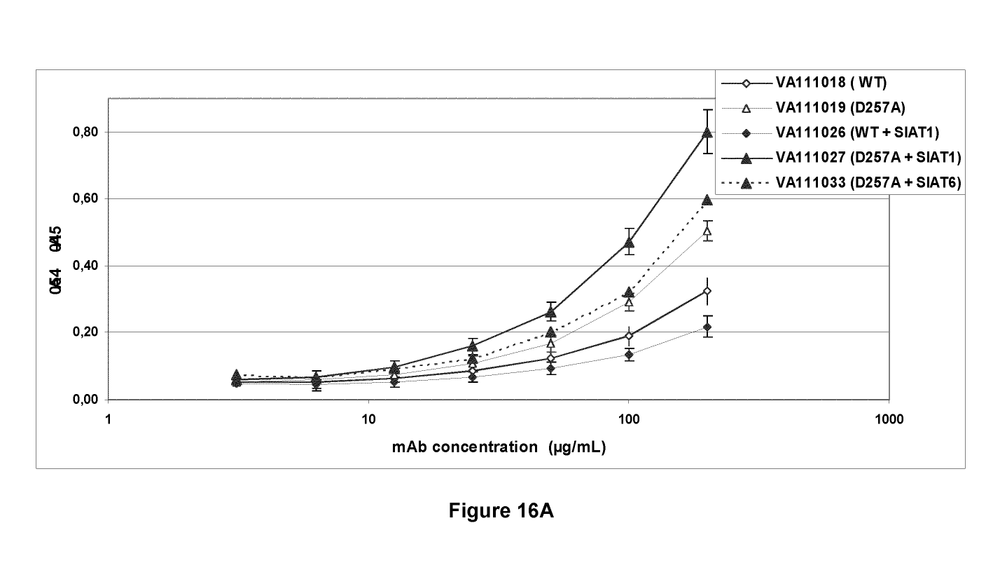

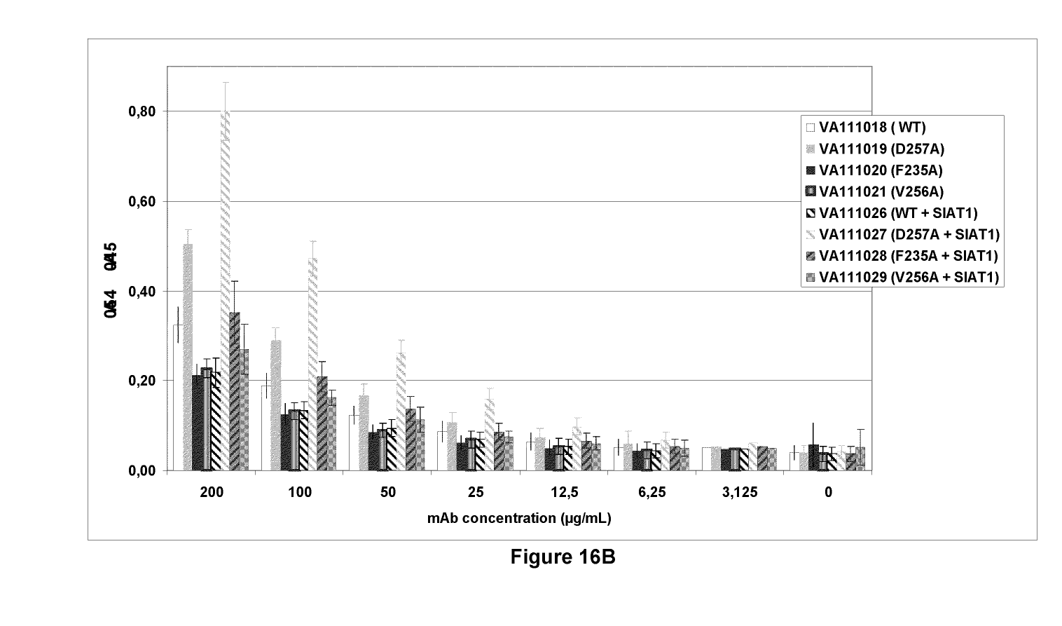

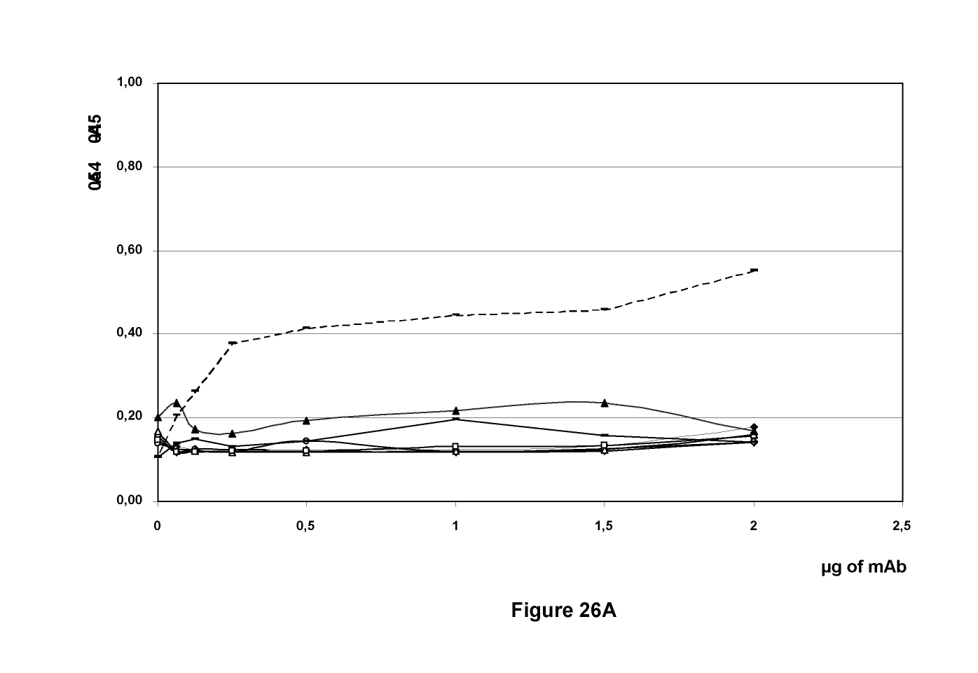

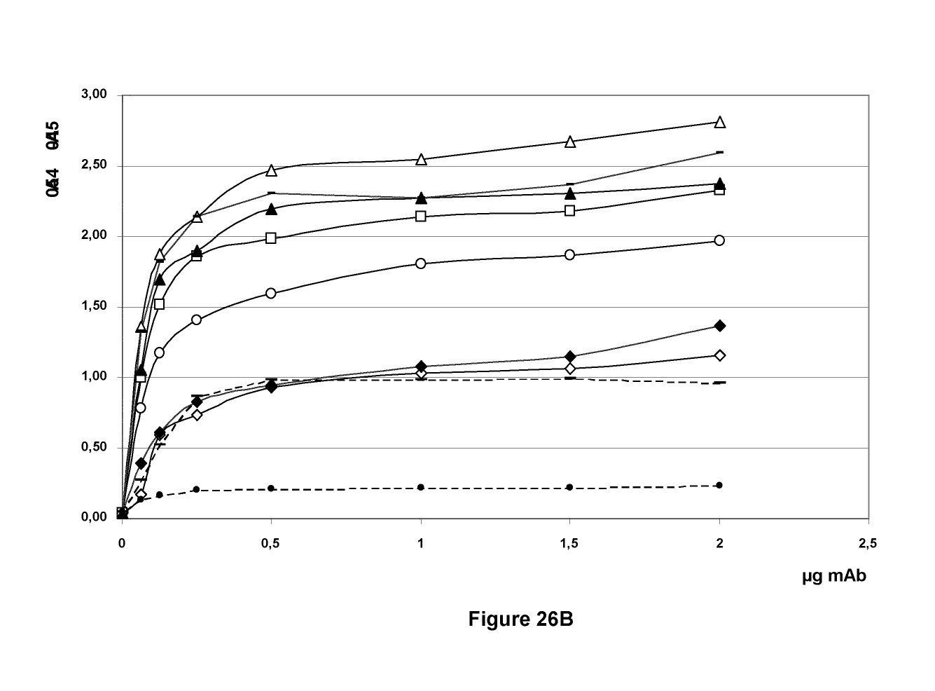

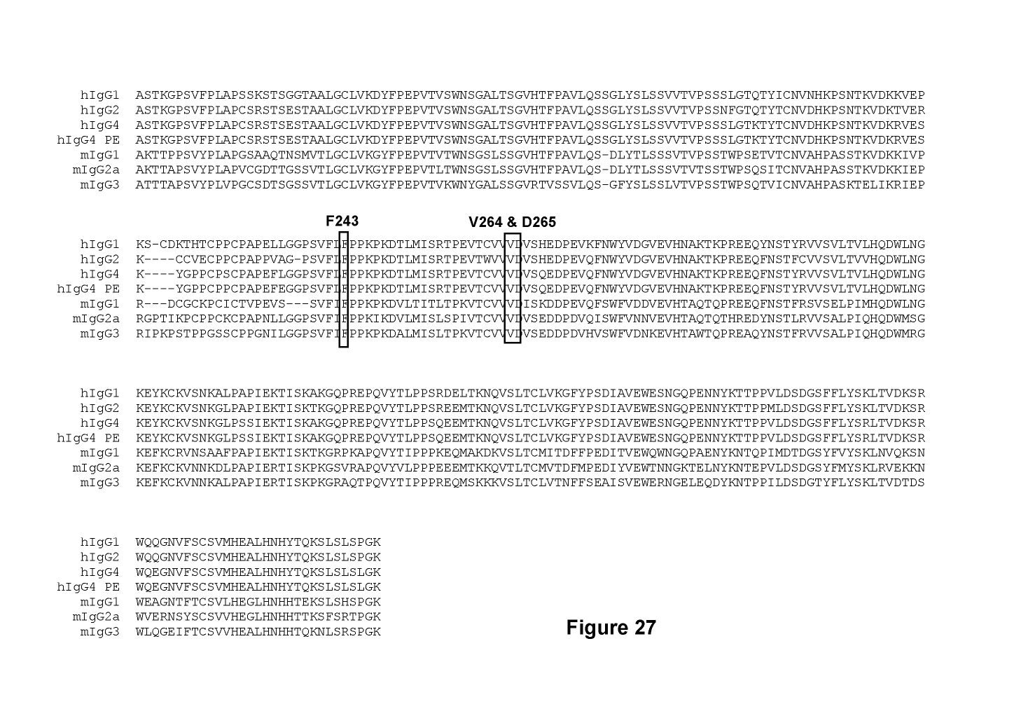

Confirmation that the SIGN-R1 binding was specific for the α-2,6 linkage was obtained by repeating the experiment with an antiAbeta_{—}13C3_D257A mAb obtained from a cell line expressing SIAT6 (example 7, batch VA1_{—}11033). This mAb contains mixed α-2,6/α-2,3 sialylated N-glycans (see FIG. 26) and leads to an intermediate level of binding to SIGN-R1 between a mAb produced in a cell line expressing B4GT1 and SIAT1, thus carrying oligosaccharides wherein most of the sialyl residues are linked to the galactoses by α-2,6 linkage (see FIG. 26), and a mAb produced in a cell line not expressing any further glycosyltransferases (FIG. 16 A.

Therefore the α-2,6 sialylated N-glycans Fc is involved in the reactivity of the mAb towards SIGN-R1.

Finally, it was investigated whether binding to SIGN-R1 was influenced by the position of the mutation in the CH₂ domain, F at 235, V at 256 or D257 on the α-2,6 sialylated antiAbeta_{—}1303. As shown on FIG. 16B, substitution at that position 257 resulted in a much increased binding.

A mutation at position 257 is thus particularly preferred, since it not only results in a fully sialylated antibody, but also to an antibody that exhibits increased binding to its target.

### Example 7

### Obtention and Characterization of α-2,6 Sialylated Humanized AntiAbeta_{—}13C3_IgG4-D260X (X=A, D, K, S, N, L, G

This example provides a method for producing α-2,6 sialylated mAbs with a human IgG4 isotype and containing a point mutation in the Fc at position 265 in the EU nomenclature. It corresponds to aspartic acid at position 260 for the corresponding position in AntiAbeta_{—}13C3_IgG4, wherein the residues are numbered from the first of the secreted mAb heavy chain.

In order to verify that the method of the invention could be applied to humanized or human antibodies, 6 different substitutions were inserted at position D260 of the IgG4 Fc domain of the humanized AntiAbeta 13C3 mAb by PCR. The amino acid introduced were A, D, K, S, N, L or G. Each of the resulting mutant antibodies was produced in HEK293 by transient expression and analyzed for its sialic acid content and its capacity to bind the SNA lectin.

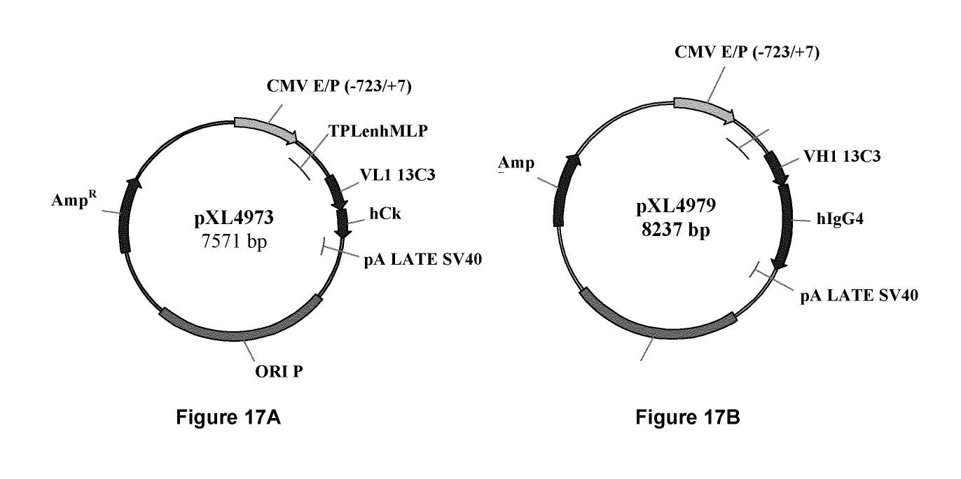

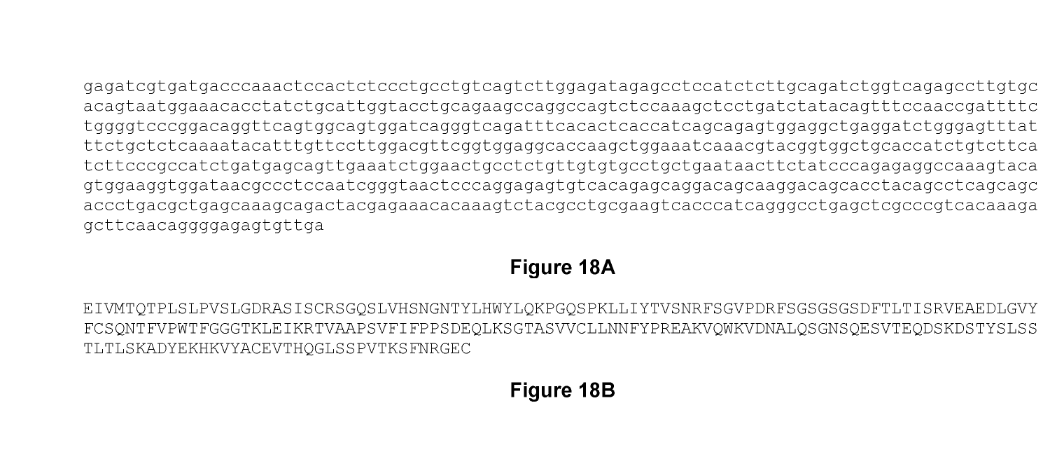

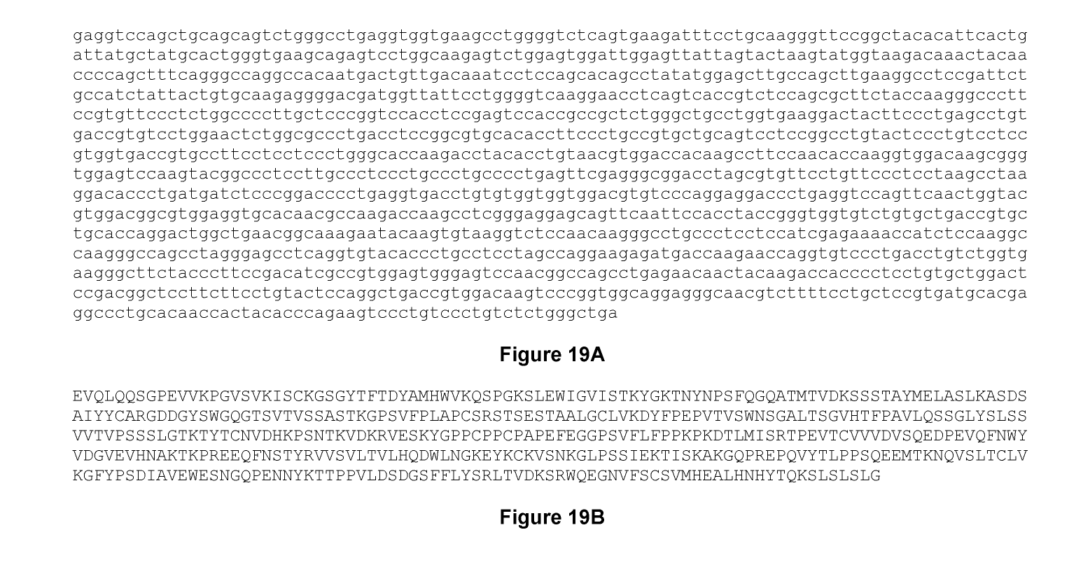

Plasmid pXL4973 encoded the humanized VL1 domain fused to human Ckappa domain (FIG. 17A), while plasmid pXL4979 encoded the humanized VH1 fused to human IgG4 constant domain of antiAbeta_{—}13C13_IgG4 mAb, FIG. A2.

The same expression vector was used to clone the cDNA encoding humanized LC and HC of AntiAbeta_{—}13C3_D260X mAb variants.

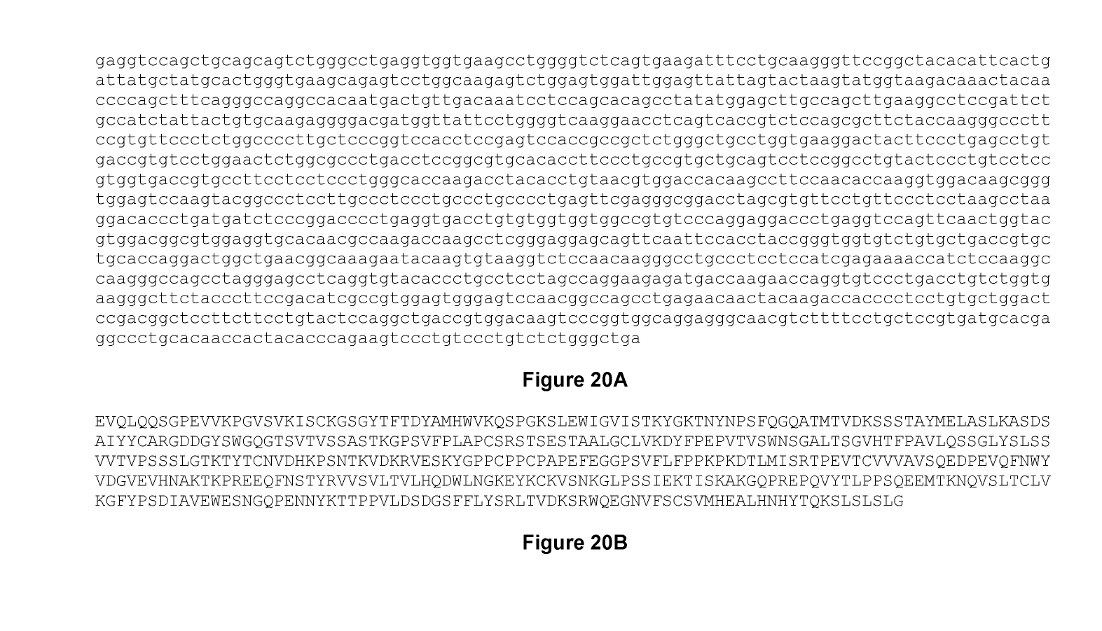

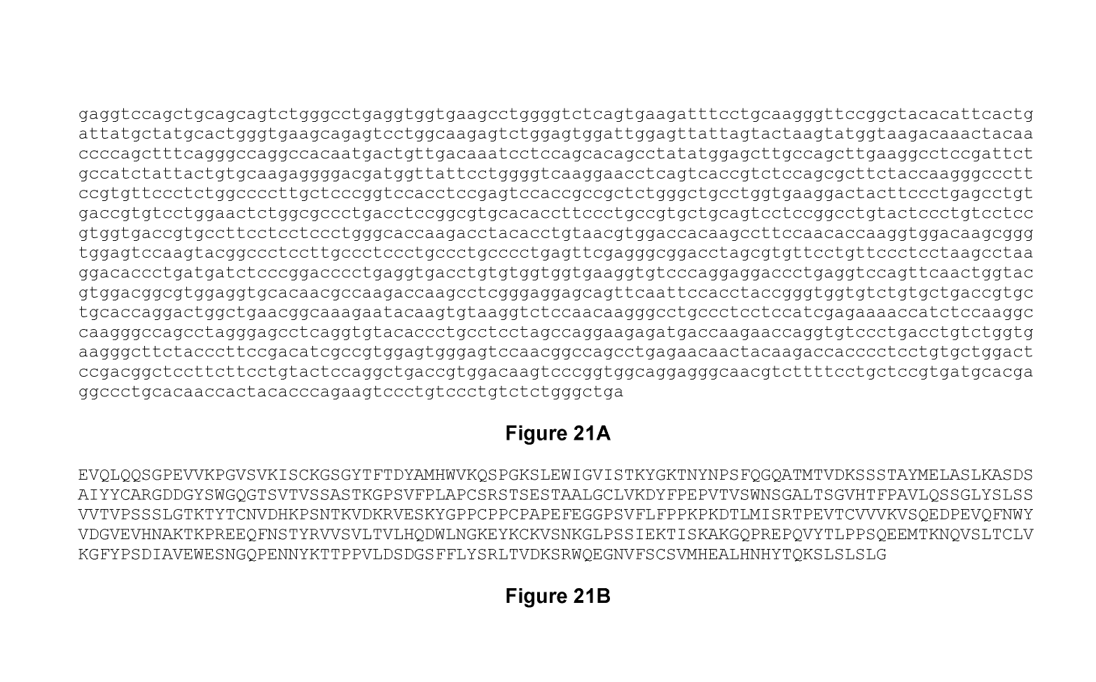

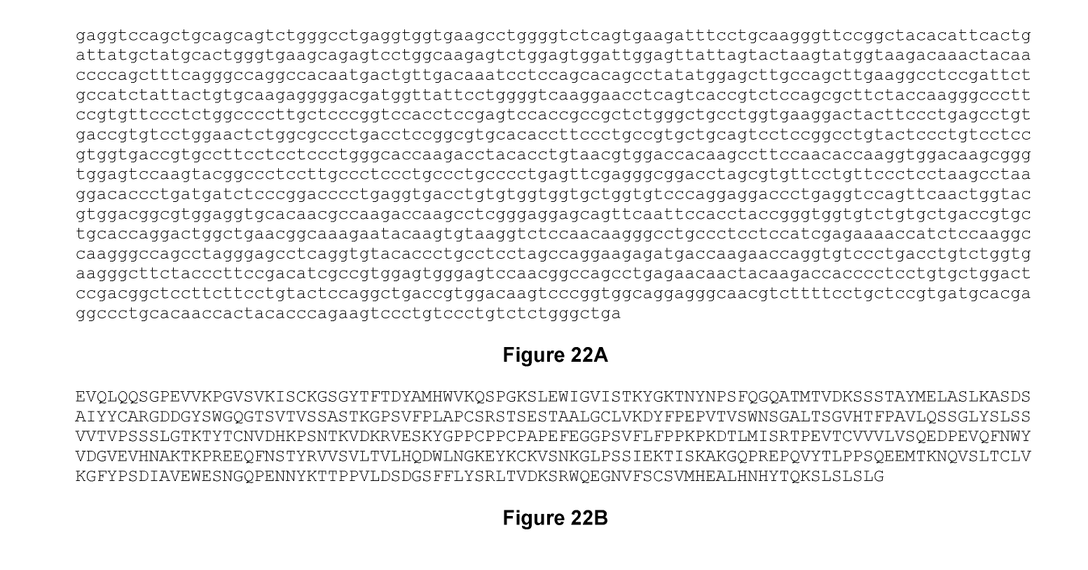

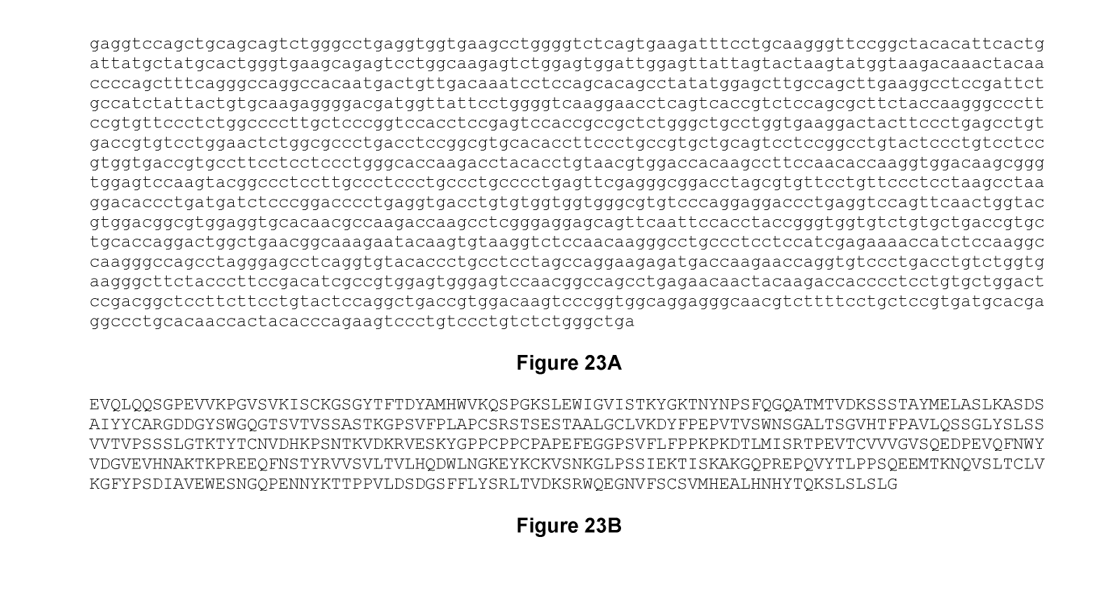

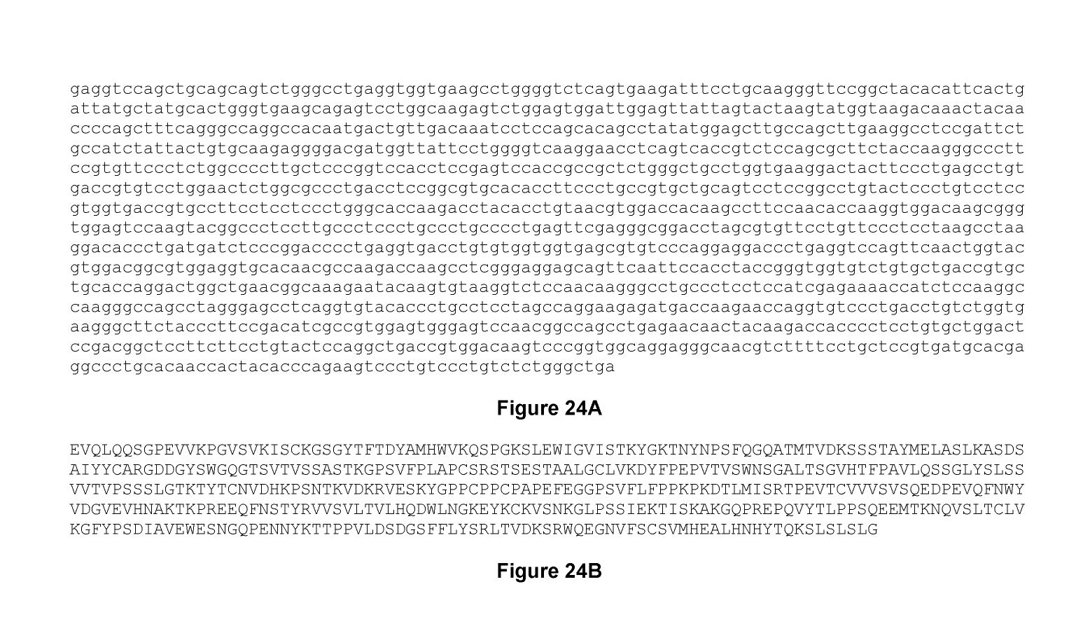

Plasmids 5227 to 5232 derived from pXL4979 by a point mutation in the IgG4 domain. Plasmid pXL5227 encoded the modified HC of AntiAbeta_{—}13C3_IgG4-D260A, Plasmid pXL5228 encoded the modified HC of AntiAbeta_{—}13C3_IgG4-D260K mAb, Plasmid pXL5229 encoded the modified HC of AntiAbeta_{—}13C3_IgG4-D260L mAb, Plasmid pXL5230 encoded the modified HC of AntiAbeta_{—}13C3_IgG4-D260G mAb, Plasmid pXL5232 encoded the modified HC of AntiAbeta_{—}13C3_IgG4-D260S mAb, The nucleic acid and corresponding amino acid sequences of the LC and HC mAb variants were described on FIGS. 20 to 24.

Each monoclonal antibody variant was produced in suspension-cultivated 293-F cells by transient co-expression of four plasmids encoding the HC, LC, SIAT1 and B4GT1 complexed with 293Fectin™ (Invitrogen). Plasmid ratio was optimized to ensure optimal productivity and sialic acid content. Optimal plasmid ratio was 6/0.5/0.5 for [HC and LC plasmids]/[SIAT1 plasmid]/[B4GT1 plasmid].

Secreted mAbs were produced with productivity ranging from 39 to 43 mg/L harvested eight days post transfection and centrifuged. MAbs were purified by affinity chromatography on Protein A (MabSelect, GE, Healthcare) and eluted from the column with 100 mM acetic acid pH 2.8, 20 mM NaCl buffer. They were formulated in PBS, 0.22 μm-filtered and stored at +5° C. Purified mAb concentrations were determined by measurement of absorbance at 280 nm.

Around 10-11 mg of mAb was purified from 500 mL culture. Each batch was analyzed by SDS-PAGE (Nupage Bistris/MOPS-SDS 4-12%, Invitrogen) under reducing and non-reducing conditions to determine a purity of more than 97% and the expected molecular weight of each subunit and of the monomer. Each batch was also analyzed by gel filtration (Tricorn 10/300 GL Superdex 200) to determine the homogeneity of the monomer and the content of high molecular weight species of less than 10%.

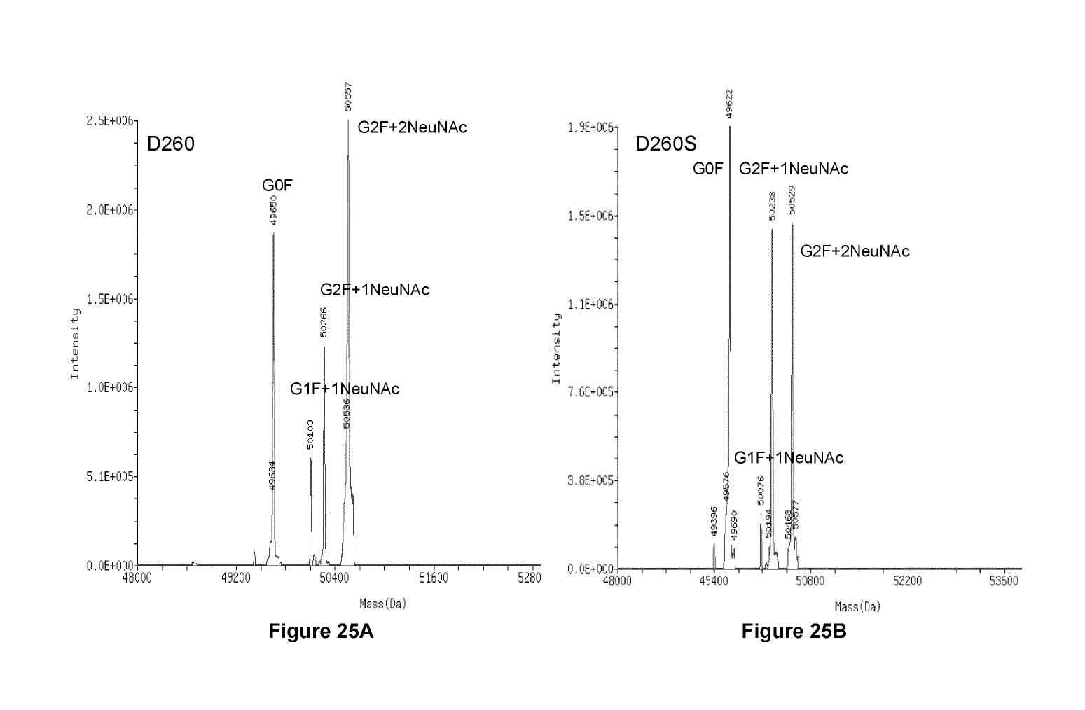

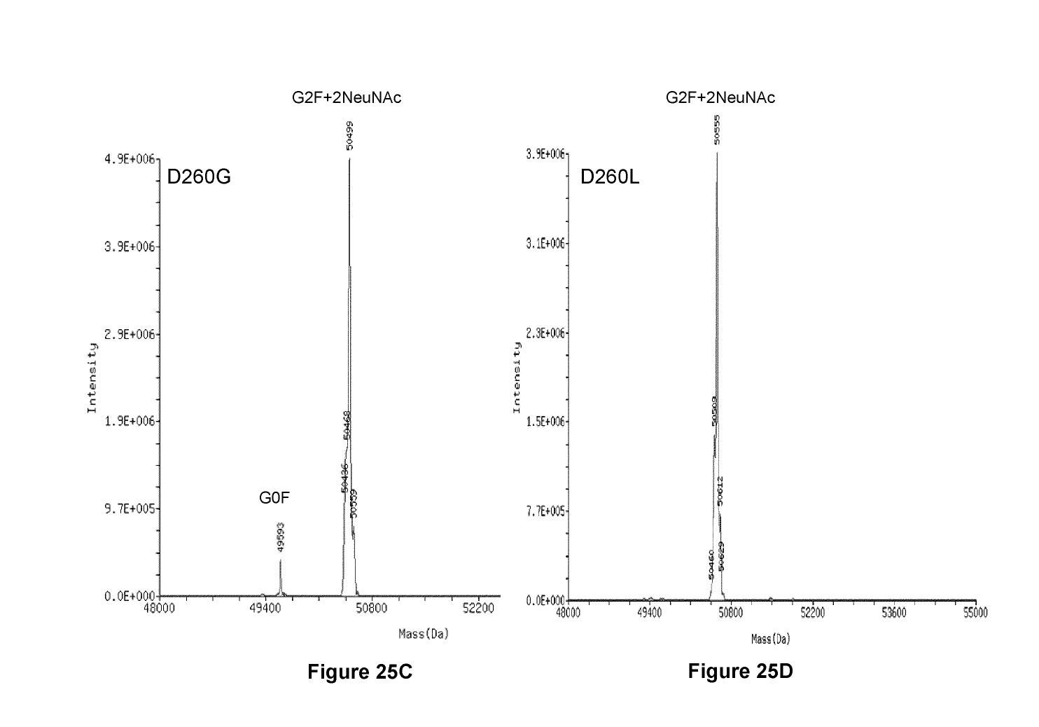

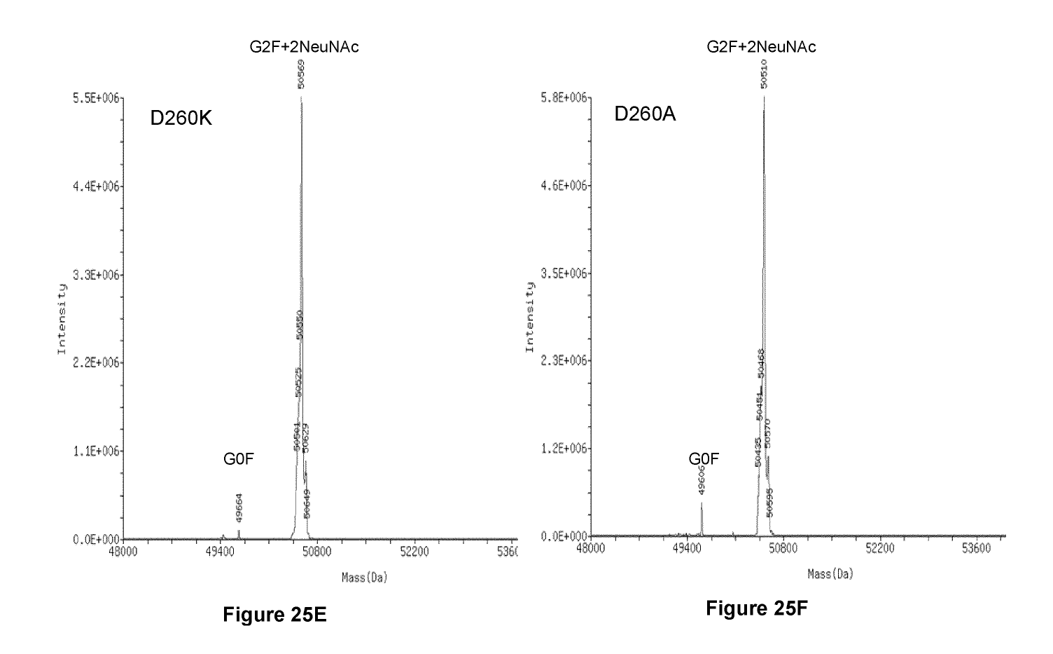

Mass spectrometry analysis was carried out on nanoLC coupled to LTQ-Orbitrap MS. It revealed the expected mass of the different mAbs for all the batches. In addition, the N-glycans were essentially sialylated with the following batches (VA1_{—}11053 to VA1_{—}11056). These batches respectively corresponded to variants containing the following point mutation in the Fc domain: D265G, D265L, D265K and D265A using the EU nomenclature (see FIG. 25 and Table 8).

Two enzyme-linked lectin assays (ELLA) were developed to detect either terminal α-2,3 sialic acid in N-glycan with lectin Maackia amurensis (MAA) or terminal α-2,6 sialic acid in N-glycan with lectin Sambucus nigra (SNA). A control batch (VA1_{—}11033) containing α-2,3 and α-2,6 sialylated AntiAbeta_{—}13C3_D257A was also included. It was produced by co-expression of the four plasmids encoding the HC pXL5105, LC pXL4808, SIAT6, pXL4544 and B4GT1 pXL4551 and purified as above.

As shown on FIG. 26, no reactivity was found to MAA with batches VA1_{—}11051 to VA1_{—}11056, whereas specificity was observed with SNA and reactivity was higher when the sialylated content of the N-glycan was higher. The ranking of the batches and the point mutation in the Fc was the following: VA1_{—}11054 VA1_{—}11056 VA1_{—}11055>VA1_{—}11053>>VA1_{—}11052-VA1_{—}11051; this translates, for the point mutations, as follows: L˜A˜K>G>>S˜D. This ranking correlates with the sialic acid content of the N-glycan of the various mutants.

TABLE 8
Characteristics of mAb variants with α-2,6-sialylated N-glycan in Fc

| | | | | Mass Spectrometry | | | |
|---|---|---|---|---|---|---|---|
| Mutation | | | | | Theoretical | | |
| (location | | Plasmids | mAb | | mass of mAb | Reactivity towards | |
| on the | | LC and | purified | Mass | with N-glycan | MAA | SNA |
| antibody) | Batch | HC | (mg) | (Da) | as | α-2,3 | α-2,6 |
| Wild- | VA1_11051 | pXL4973 | 11.2 | 49650 | G0F | no | intermediate |
| type | | pXL4979 | | 50103 | G1F + 1 | | |
| | | | | 50266 | NeuNAc | | |
| | | | | 50557 | G2F + 1 | | |
| | | | | | NeuNAc | | |
| | | | | | G2F + 2 | | |
| | | | | | NeuNAc | | |
| D260S | VA1_11052 | pXL4973 | 10.0 | 49622 | G0F | no | intermediate |
| | | pXL5232 | | 50076 | G1F + 1 | | |
| | | | | 50238 | NeuNAc | | |
| | | | | 50529 | G2F + 1 | | |
| | | | | | NeuNAc | | |
| | | | | | G2F + 2 | | |
| | | | | | NeuNAc | | |
| D260G | VA1_11053 | pXL4973 | 11.0 | 50499 | G2F + 2 | no | high |
| | | pXL5230 | | (major) | NeuNAc | | |
| | | | | 49593 | G0F | | |
| | | | | (minor) | | | |
| D260L | VA1_11054 | pXL4973 | 10.0 | 50555 | G2F + 2 | no | high |
| | | pXL5229 | | | NeuNAc | | |
| D260K | VA1_11055 | pXL4973 | 10.8 | 50569 | G2F + 2 | no | high |
| | | pXL5228 | | (major) | NeuNAc | | |
| | | | | 49664 | G0F | | |
| | | | | (minor) | | | |
| D260A | VA1_11056 | pXL4973 | 10.2 | 50510 | G2F + 2 | no | high |
| | | pXL5227 | | (major) | NeuNAc | | |
| | | | | 49606 | G0F | | |
| | | | | (minor) | | | |

In conclusion, D265A, D265G, D265L and D265K mutations all lead to an enhanced proportion of disialylated antibody molecules.

1. A method for producing an IgG antibody, wherein at least 80% of the said antibody comprises a complex, bi-antennary oligosaccharide, which contains two sialic acid residues, attached to each Fc domain of the antibody, said method comprising the steps of:
a) introducing a mutation in the said Fc domain of the said antibody, and
b) expressing the mutant antibody obtained in step a) in a cell line expressing a β-galactosyltransferase and a sialyltransferase activity.

2. The method of claim 1, wherein the β-galactosyltransferase is a β-1,4-galactosyltransferase and the sialyltransferase is a α-2,6-sialyltransferase.

3. The method of claim 1, wherein the β-1,4-galactosyltransferase is encoded by the polynucleotide sequence represented by SEQ ID NO: 35 and the α-2,6-sialyltransferase is encoded by the polynucleotide sequence represented by SEQ ID NO: 33.

4. The method of claim 1, wherein the said sialic acid residues are linked to the antibody through an α-2,6-linkage.

5. The method of claim 1 wherein the antibody is a monoclonal antibody.

6. The method of claim 1, wherein the antibody is a humanized antibody.

7. The method of claim 1, wherein the said mutation affects an amino acid selected from the group consisting of F243, V264, and D265.

8. The method of claim 1, wherein the said mutation is a substitution of the said amino acid by an amino acid selected from the group consisting of alanine (A), glycine (G), leucine (L), and lysine (K).

9. The method of claim 1, wherein the said mutation is selected from the group consisting of D265L, D265K, and D265A.

10. The method of claim 1, wherein the said antibody comprises a human IgG4 Fc domain.

11. The method of claim 1, wherein the said antibody comprises a human IgG1 Fc domain.

12. The method of claim 1, wherein said cell line expressing a β-galactosyltransferase and a sialyltransferase activity is a cell line that is stably transfected with one or two vectors encoding beta-galactosyltransferase and sialyltransferase.

13. The method of claim 1, wherein said cell line expressing a β-galactosyltransferase and a sialyltransferase activity is a cell line that is stably transfected with one or two vectors encoding said antibody.

14. An antibody produced by the method of claim 1.

15. A pharmaceutical composition comprising the antibody of claim 14.

16. (canceled)

17. A composition comprising an IgG antibody, wherein at least 80% of the said antibody comprises a complex, bi-antennary oligosaccharide attached to each Fc domain of the said antibody, said oligosaccharide comprising two sialic acid residues, wherein the Fc domain comprises an amino sequence which differs from a native sequence human IgG Fc domain.

18. The composition of claim 17 wherein the said sialic acid residues are linked to the antibody through an α-2,6-linkage.

19. The composition of claim 17, wherein the antibody of the composition of the invention comprises an amino acid substitution at any one or more of amino acid positions 243, 264 and 265.

20. The composition of claim 19, wherein the said substitution is a substitution of the said amino acid by an amino acid selected from the group consisting of alanine (A), glycine (G), leucine (L), and lysine (K).

21. The composition of claim 20, wherein the said substitution is selected from the group consisting of D265L, D265K, and D265A.
